(19) United States
(12) Patent Application Publication
(10) Pub. No.: US 2025/0243507 A1
(43) Pub. Date: Jul. 31, 2025

(54) INTERNAL RIBOSOME ENTRY SITE (IRES), PLASMID VECTOR AND CIRCULAR MRNA FOR ENHANCING PROTEIN EXPRESSION

(71) Applicants: BIO ADVENTURE CO., LTD., Bangkok (TH); MAHIDOL UNIVERSITY, Phuttamonthon Nakornpathom (TH); OFFICE OF NATIONAL HIGHER EDUCATION SCIENCE RESEARCH AND INNOVATION POLICY COUNCIL, Patumwan Bangkok (TH)

(72) Inventors: Patompon WONGTRAKOONGATE, Bangkok (TH); Suradej HONGENG, Bang Phlat, Bangkok (TH)

(73) Assignees: BIO ADVENTURE CO., LTD., Bangkok (TH); MAHIDOL UNIVERSITY, Phuttamonthon Nakornpathom (TH); OFFICE OF NATIONAL HIGHER EDUCATION SCIENCE RESEARCH AND INNOVATION POLICY COUNCIL, Patumwan Bangkok (TH)

(21) Appl. No.: 18/848,164

(22) PCT Filed: Mar. 21, 2023
(86) PCT No.: PCT/TH2023/000003

§ 371 Date: Sep. 18, 2024

(30) Foreign Application Priority Data
Mar. 21, 2022 (TH) 2201001686

(51) Int. Cl. C12N 15/85; C07K 14/005; C07K 14/47; C07K 14/705

(52) U.S. Cl. CPC C12N 15/85; C07K 14/005; C07K 14/4748; C07K 14/70503; C12N 2770/20022; C12N 2840/203

(57) ABSTRACT

The present invention relates to a plasmid vector for making an open reading frame-coding circular mRNA (ORF-coding circular mRNA) having a small backbone, optimized homology arms and internal ribosome entry site (IRES) that encodes an efficient open reading frame (ORF), resulting in highly efficient protein expression. The IRES has also been developed to promote the translation of proteins of interest, thus improving protein expression. The plasmid vector and ORF-coding circular mRNA according to this invention can overcome previous technical challenges due to its optimized constructs and small size, resulting in ease of delivery, stability in the cell, and significantly higher translation efficiency and protein expression in vivo. When applied in medicine or pharmaceutical preparations, the circular mRNA described in the present invention can potentially reduce the frequency of administration and/or doses required, resulting in fewer unwanted side effects and improved patient access to medicines.

### FIELD OF INVENTION

This invention relates to biotechnology, more particular, to an internal ribosome entry site (IRES), plasmid vector and circular mRNA for enhancing protein expression.

### BACKGROUND OF THE INVENTION

Genetic engineering has become a valuable tool for developing targeted prevention and treatment approaches. This technique involves using genetic materials in organisms' cells to control protein synthesis specific to disease pathology. One area of focus has been messenger ribonucleic acid (mRNA), which is a single-stranded molecule that is complementary to the template gene's deoxyribonucleic acid (DNA). mRNA is made up of nucleotides arranged in a linear sequence. During protein synthesis, ribosomes bind to mRNA to read the nucleotide sequences, which are then translated into proteins.

The advantages of mRNA in medicine are significant. It is relatively easy to synthesize mRNA in vitro within a short period, and it can activate rapid responses when administered to organisms, allowing for targeted delivery and reducing the risk of unwanted side effects. mRNA can be easily synthesized in a laboratory within a short period and can activate a rapid response when administered to organisms. Moreover, mRNA does not pose any concerns about integration into the organism's genome when applied for medical purposes, unlike plasmid vectors.

While the mRNA technique in medicine offers numerous advantages, it has a drawback in that mRNA is inherently unstable and susceptible to degradation by cellular mechanisms when administered to living organisms. Consequently, mRNA is only suitable for a limited period of use in activating rapid cell response. To overcome these technical challenges, researchers have been exploring methods to extend the activity of mRNA to enhance protein expression efficiency and stability.

The US Patent Publication No. 20200080106 A1 disclosed a circular RNA developed to increase the efficiency of disease treatment. A vector for constructing circular RNA, said vector comprising various elements connected to each other and arranged in a following sequence including a 5′ homology arm connected to a 3′ group I intron fragment, 5′ spacer, internal ribosome entry site (IRES), protein-coding region, 5′ group I intron fragment, and 3′ homology arm. The circular RNA can be translated to protein or biologically active inside eukaryotic cells and can be delivered into cells by transfection. The disclosure showed that circular RNA can produces greater protein expression for a longer duration in cells, leading to higher treatment efficiency. However, RNA circularization and IRES of the disclosed circular RNA have not been optimized. Therefore, the disclosed circular RNA results in lower protein expression. Moreover, expression of various IRES used by the disclosed circular RNA was not tested in mice.

The European Patent No. EP 2996697 B1 disclosed a high stability circular RNA with efficiently translate proteins of interest inside eukaryotic cells, making it useful for therapeutics. It is well established that circular RNA molecules have much longer half-life than their linear RNA molecules. This document suggested increasing RNA half-life and stability by RNA circularization, which reduces excretion by exonuclease when delivered into cells. The disclosure revealed that the circular RNA half-life is approximately 40 hours in vivo, which is higher than the linear mRNA half-life of only 6 to 8 hours. Again, expression of the disclosed circular RNAs using different IRES was not tested in mice.

The International Patent Publication No. WO 2020237227 A1 disclosed circular RNA with high stability to control gene expression in organisms, which can be applied in disease treatment such as gene therapy or vaccines. This document also disclosed an internal ribosome entry site (IRES) modification using viral components such as salivirus A SZ1, salivirus A BN2, and coxsackievirus B3 (CVB3). It was found that modifying the IRES with viral components such as salivirus A SZ1 and salivirus A BN2 provided circular RNA with high functional stability. However, according to the disclosure, the RNA stability is not ideal, as the protein expression level decreases over time.

As demonstrated by the above disclosures, despite the advantages of circular RNA, its size and limited IRES activity may impede efficient translation in vivo. In addition, the circularization can also be further improved.

Biotechnology Journal, 2014, 9 (9), 1164-1174, disclosed that co-transfection of plasmid vectors encoding fusion protein and plasmid vectors encoding Bcl-xL protein into CHO cells significantly increased fusion protein expression and provided cell stability for more than six days. However, the co-transfection process used DNA plasmid vectors.

Therefore, the present invention is to develop a plasmid vector for making an open reading frame-coding circular mRNA (ORF-coding circular mRNA) with a small backbone having assisted homology arms to facilitate RNA circularization. The internal ribosome entry site (IRES) has also been developed to enhance translation of proteins of interest, thus improving protein expression. The plasmid vector and ORF-coding circular mRNA according to this invention can overcome previous technical challenges due to its optimized constructs and small size, resulting in significantly higher translation efficiency and protein expression in vivo.

### SUMMARY OF THE INVENTION

The present invention relates to a plasmid vector for making an open reading frame-coding circular mRNA (ORF-coding circular mRNA) having a small backbone, optimized homology arms that encodes an efficient open reading frame (ORF), resulting in highly efficient protein expression. The internal ribosome entry site (IRES) has also been developed to promote translation of proteins of interest, thus improving protein expression in vivo. The plasmid vector and ORF-coding circular mRNA according to this invention can overcome previous technical challenges due to its optimized constructs and small size, resulting in significantly higher translation efficiency and protein expression in vivo. When applied in medicine or pharmaceutical preparations, the circular mRNA described in the present invention can potentially reduce the frequency of administration and/or doses required, resulting in fewer unwanted side effects and improved patient access to medicines.

### BRIEF DESCRIPTION OF THE DRAWINGS

FIG. 1 shows an example of circular mRNA constructs according to this invention.

FIG. 2 shows the flow cytometry histograms of HEK293T cells at approximately 48 hours after transfection with the circular GFP mRNA (CVB3 IRES) using various commercially available delivery systems.

FIG. 3 shows the fluorescent intensity observed in HEK293T cells at approximately 48 hours after transfection with the circular GFP mRNA (CVB3 IRES) using various commercially available delivery systems.

FIG. 4 shows the luciferase activity observed in the supernatant of HEK293T cells at approximately 24 hours after transfection with the circular luciferase mRNA (CVB3 IRES) by using various commercially available delivery systems.

FIG. 5 shows the luciferase activity observed in the supernatant of HEK293T cells approximately at 48 hours after transfection with the circular luciferase mRNA (CVB3 IRES) by using various commercially available delivery systems.

FIG. 6 shows flow cytometry histograms of HEK293T cells at approximately 48 hours after transfection with the circular GFP mRNA containing different types of IRES.

FIG. 7 shows the percentage of HEK293T cells exhibiting positive GFP at approximately 48 hours after transfection with the circular GFP mRNA containing different types of IRES.

FIG. 8 shows the luminescent image in mice (BALB/c species) at approximately 24 and 48 hours after injection of the circular luciferase mRNA (CVB3 IRES) via the delivery system of sample B by intramuscular (I.M.) and intravenous (I.V.) injection.

FIG. 9 shows flow cytometry histograms of HEK293T cells at approximately 48 hours after transfection with the circular SARS-CoV-2 spike protein mRNA.

FIG. 10 shows results of the western blot analysis to detect the expression of spike protein of the SARS-CoV-2 virus in the supernatant obtained from HEK293T cells after transfection with the circular SARS-CoV-2 spike protein mRNA.

FIG. 11 shows the analysis of the expression of immunoglobulin G (IgG) expression specific to the spike protein of the SARS-CoV-2 virus Omicron species (B.1.1.529) in mice serum at approximately 2 weeks after injection of the circular mRNA (CVB3 IRES) that expresses the spike protein, compared to the control mice, using an enzyme-linked immunosorbent assay (ELISA).

FIG. 12 shows the inhibitory capacity of pseudovirus particle invasion of the SARS-CoV-2 virus Omicron species (B.1.1.529) with mice serums measured using a neutralization assay at approximately 2 weeks after injection of the circular mRNA (CVB3 IRES) that expresses the spike protein, compared to the control mice.

FIG. 13 shows the fluorescent image of HEK293T cells at approximately 48 hours after transfection with the circular cancer antigen protein mRNA H3K27M, using an immunofluorescence assay.

FIG. 14 shows the fluorescent image of HEK293T cells at approximately 48 hours after transfection with the circular cancer antigen protein mRNA PSCA, using an immunofluorescence assay.

FIG. 15 shows the fluorescent image of HEK293T cells at approximately 48 hours after transfection with the circular cancer antigen protein mRNA TROP2, using an immunofluorescence assay.

FIG. 16 shows the fluorescent image of HEK293T cells at approximately 48 hours after transfection with the circular reprogramming factor mRNA OSCK, using an immunofluorescence assay by gram staining with antibodies specific to the OCT4 protein.

FIG. 17 shows the fluorescent image of HEK293T cells at approximately 48 hours after transfection with the circular reprogramming factor mRNA OSCK, using an immunofluorescence assay by gram staining with antibodies specific to the SOX2 protein.

FIG. 18 shows the fluorescent image of HEK293T cells at approximately 48 hours after transfection with the circular CAR mRNA CD19, using an immunofluorescence assay.

FIG. 19 shows in vitro transcribed (IVT), circularized products (IC), and IC treated with RNase R compared between circular RNA-eHA-1 and circular RNA-eHA-2 using an agarose gel electrophoresis. The white arrow indicates the circular form of RNA.

FIG. 20 shows the luciferase activity compared between two different designed external homology arms: circular Fluc-eHA-1-CVB3 and circular Fluc-eHA-2-CVB3 in HEK293T cells. Statistical significance was determined with unpaired t-test. Mean±SEM; n=2; *p<0.05.

FIG. 21 shows the secondary structures of IRES sequences predicted using the online tool RNAfold. Only the secondary structures of the IRES derived from CVB3, ECH20, chimeric 3 (20) 3, and chimeric 20 (3) 20 are shown here.

FIG. 22 shows the mean fluorescence intensity (MFI) and the difference in mean fluorescence intensity (ΔMFI) of GFP in the imHC cells between different IRES. Statistical significance was determined with an unpaired t-test. Mean±SEM; n=3; *p<0.05; **p<0.01.

FIG. 23 shows the mean fluorescence intensity (MFI) and difference in mean fluorescence intensity (ΔMFI) of GFP in BHK-21 cells between different IRES in the BHK-21 cells.

FIG. 24 shows in vivo bioluminescence images taken at (A) 6 hours and (B) 48 hours after intramuscular (I.M.) injection of the circular Fluc-eHA-2-CVB3 or circular Fluc-eHA-2-3 (20) 3 encapsulated with LNP in BALB/c mice. A negative control group was administered with PBS.

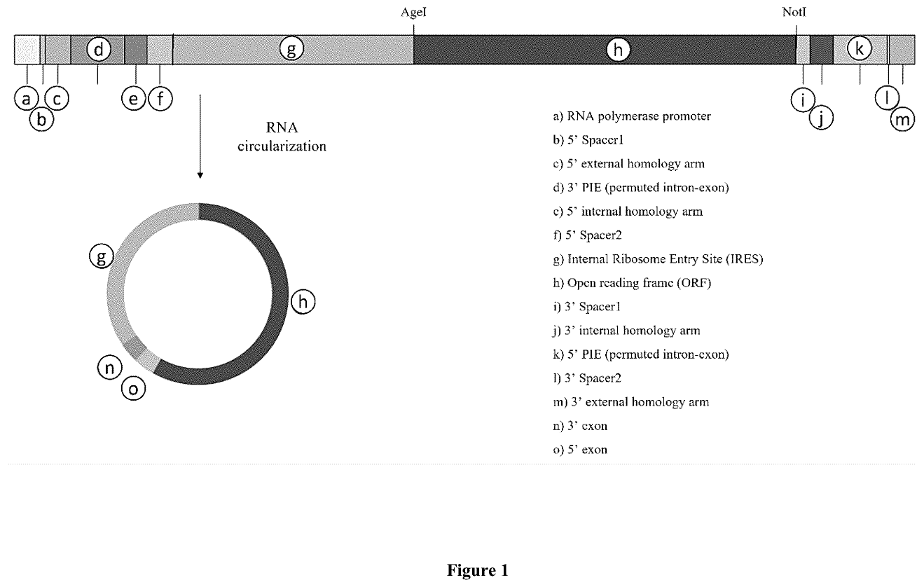

### DETAILED DESCRIPTION OF THE INVENTION

The present invention relates to an ORF-coding circular mRNA with a small backbone, optimized homology arms and IRES for highly efficient protein expression in vivo. According to the present invention, the IRES has been developed to promote the translation of proteins of interest, thus improving protein expression. As a result, the plasmid vector and ORF-coding circular mRNA according to this invention can overcome previous technical challenges due to its small size and optimized constructs, resulting in ease of delivery, stability in the cell, and significantly higher translation efficiency and protein expression in vivo. As such, when apply the circular mRNA of the present invention in medicine or pharmaceutical preparations, it can reduce the frequency of administration and/or doses required, potentially reducing unwanted side effects and increasing patient access to medicines.

As will be appreciated by those skilled in the art, the below aspects of the invention can, and preferably do, include any one or more or all of the preferred and optional features of the invention disclosed herein, as applicable and appropriate. Furthermore, a skilled person would recognize that features and/or elements mentioned in the disclosure may be altered by other features and/or elements without departing from the scope of the disclosure, even if they are not explicitly stated herein. For instance, other protein-encoding ORFs could be used to make the ORF-coding circular mRNA that correspond to its relevant functions, instead of ORFs mentioned in the disclosure. Accordingly, the scope of this disclosure should not be limited to any particular types of ORFs.

### Definition

This invention contains a Sequence Listing which is presented in XML file format and submitted via the electronic filing system.

Technical terms or scientific terms used herein have definitions as understood by those having ordinary skills in the art unless stated otherwise.

Equipment, apparatus, methods, or chemicals mentioned here refer to those commonly operated or used by those skilled in the art, unless explicitly stated otherwise, that they are equipment, apparatus, methods, or chemicals specifically used in this invention.

The use of singular or plural nouns with the term “comprising” in the claims or the specification should be interpreted as “one” as well as “one or more,” “at least one,” and “one or more than one.”

All compositions and/or processes disclosed and claimed are intended to encompass aspects of the invention that involve actions, operation, modifications, or changes of any parameters without significantly deviating from experiments performed, examples described, or data shown in this invention, and obtaining similar objects with the same utilities and results as those described in the present invention by persons skilled in the art, even without specific mention in the claims. Therefore, substitutions or similar objects to the present invention, including minor modifications or changes that are apparent to persons skilled in the art, should be considered within the scope, spirit, and concept of the invention as defined by the appended claims.

Throughout this application, the term “about” is used to indicate that any value presented herein may potentially vary or deviate due to variety of factors, such as calculation errors, discrepancies in apparatus or methods, or differences between individual operators implementing the apparatus or methods.

According to a first embodiment, the invention relates to an Internal Ribosome Entry Site (IRES) comprising a nucleotide sequence selected from the group consisting of SEQ ID NO: 21, SEQ ID NO: 22, SEQ ID NO: 23, SEQ ID NO: 24, SEQ ID NO:25 or SEQ ID NO: 26 or a combination thereof.

In a preferred exemplary embodiment, the IRES comprises the nucleotide sequence as show in SEQ ID NO: 21.

According to a second embodiment, the invention relates to a plasmid vector for making an open reading frame-coding circular mRNA (ORF-coding circular mRNA), said vector comprising elements that are connected to each other and arranged in the following sequence:
RNA polymerase promoter,
5′ Spacer1,
5′ external homology arm,
3′ PIE (permuted intron-exon),
5′ internal homology arm,
5′ Spacer2,
Internal Ribosome Entry Site (IRES),
Open reading frame (ORF),
3′ Spacer1,
3′ internal homology arm,
5′ PIE (permuted intron-exon),
3′ Spacer2, and
3′ external homology arm

In an exemplary embodiment of the invention, the plasmid vector for making the ORF-coding circular mRNA comprises the RNA polymerase promoter having a length ranging from 15 to 25 nucleotides.

In an exemplary embodiment of the invention, the RNA polymerase promoter is selected from the group consisting of T7 virus RNA polymerase promoter, SP6 virus RNA polymerase promoter, or T3 virus RNA polymerase.

In a preferred exemplary embodiment of the invention, the RNA polymerase promoter is the T7 virus RNA polymerase promoter.

In an exemplary embodiment of the invention, the plasmid vector for making the ORF-coding circular mRNA comprises the 5′ spacer1 having a length ranging from 5 to 15 nucleotides.

In an exemplary embodiment of the invention, the plasmid vector for making the ORF-coding circular mRNA comprises the 5′ external homology arm having a length ranging from 15 to 30 nucleotides.

In an exemplary embodiment of the invention, the plasmid vector for making the ORF-coding circular mRNA comprises the 5′ external homology arm having a length ranging from 15 to 20 nucleotides.

In an exemplary embodiment of the invention, the plasmid vector for making the ORF-coding circular mRNA comprises the 5′ external homology arm having a length ranging from 20 to 30 nucleotides.

In a preferred exemplary embodiment of the invention, the 5′ external homology arm is sequence SEQ ID NO: 4.

In an exemplary embodiment of the invention, the plasmid vector for making the ORF-coding circular mRNA comprises the 3′ PIE (permuted intron-exon) having a length ranging from 100 to 250 nucleotides.

In an exemplary embodiment of the invention, the plasmid vector for making the ORF-coding circular mRNA comprises the 3′ PIE (permuted intron-exon) obtained from Cyanobacterium anabaena pre-tRNA group I intron gene.

In an exemplary embodiment of the invention, the plasmid vector for making the ORF-coding circular mRNA comprises the 5′ internal homology arm having a length ranging from 15 to 25 nucleotides.

In an exemplary embodiment of the invention, the plasmid vector for making the ORF-coding circular mRNA comprises the 5′ spacer2 having a length ranging from 50 to 100 nucleotides.

In an exemplary embodiment of the invention, the plasmid vector for making the ORF-coding circular mRNA comprises an IRES having a length from 190 to 900 nucleotides.

In an exemplary embodiment of the invention, the IRES is selected from the group consisting of echovirus 33 IRES, echovirus 20 IRES, echovirus 29 IRES, coxsackievirus B1 IRES, coxsackievirus B3 IRES, coxsackievirus A12 IRES, enterovirus 80 IRES, Aphis glycines virus 1 IRES, halastavi árva virus IRES, or pegivirus J IRES or a combination thereof.

In a preferred exemplary embodiment of the invention, the IRES is the coxsackievirus B3 IRES.

In an exemplary embodiment of the invention, the IRES is selected from SEQ ID NO: 21, SEQ ID NO: 22, SEQ ID NO: 23, SEQ ID NO: 24, SEQ ID NO:25 or SEQ ID NO: 26 or a combination thereof.

In a preferred exemplary embodiment of the invention, the IRES is sequence SEQ ID NO: 21.

In an exemplary embodiment of the invention, the plasmid vector for making the ORF-coding circular mRNA comprising the ORF is selected from the group consisting of ORF encoding virus spike protein, ORF encoding cancer antigen protein, ORF encoding reprogramming factor protein, or ORF encoding chimeric antigen receptor protein (CAR protein) or a combination thereof.

In a preferred exemplary embodiment of the invention, the ORF encoding virus spike protein is ORF encoding SARS-CoV-2 virus spike protein.

In a preferred exemplary embodiment of the invention, the ORF encoding cancer antigen protein is ORF encoding cancer antigen protein H3K27M, ORF encoding cancer antigen protein PSCA, or ORF encoding cancer antigen protein TROP2.

In a preferred exemplary embodiment of the invention, the ORF encoding reprogramming factor protein is ORF encoding reprogramming factor protein OSCK.

In a preferred exemplary embodiment of the invention, the ORF encoding chimeric antigen receptor protein (CAR protein) is ORF encoding CAR protein CD19.

In an exemplary embodiment of the invention, the plasmid vector for making the ORF-coding circular mRNA comprises the 3′ internal homology arm having a length ranging from 15 to 25 nucleotides.

In an exemplary embodiment of the invention, the plasmid vector for making the ORF-coding circular mRNA comprises the 5′ PIE (permuted intron-exon) having a length ranging from 100 to 150 nucleotides.

In an exemplary embodiment of the invention, the plasmid vector for making the ORF-coding circular mRNA comprises the 5′ PIE (permuted intron-exon) obtained from Cyanobacterium anabaena pre-tRNA group I intron gene.

In an exemplary embodiment of the invention, the plasmid vector for making the ORF-coding circular mRNA comprises the 3′ spacer2 having a length ranging from 5 to 15 nucleotides.

In an exemplary embodiment of the invention, the plasmid vector for making the ORF-coding circular mRNA comprises the 3′ external homology arm having a length ranging from 15 to 30 nucleotides.

In an exemplary embodiment of the invention, the plasmid vector for making the ORF-coding circular mRNA comprises the 3′ external homology arm having a length ranging from 15 to 25 nucleotides.

In an exemplary embodiment of the invention, the plasmid vector for making the ORF-coding circular mRNA comprises the 3′ external homology arm having a length ranging from 25 to 30 nucleotides.

In a preferred exemplary embodiment of the invention, the 3′ external homology arm is sequence SEQ ID NO: 32.

In an exemplary embodiment of the invention, ORF-coding circular mRNA obtained from the plasmid vector according to any one of the above-mentioned embodiments of the invention.

According to a third embodiment, this invention, relates to an open reading frame-coding circular mRNA (ORF-coding circular mRNA), the ORF-coding circular mRNA comprising elements that are connected to each other and arranged in the following sequence:
3′ exon
Internal Ribosome Entry Site (IRES)
Open reading frame (ORF)
5′ exon

In an exemplary embodiment of the invention, the ORF-coding circular mRNA comprises the IRES having a length ranging from 190 to 900 nucleotides.

In an exemplary embodiment of the invention, the IRES is selected from the group consisting of echovirus 33 IRES, echovirus 20 IRES, echovirus 29 IRES, coxsackievirus B1 IRES, coxsackievirus B3 IRES, coxsackievirus A12 IRES, enterovirus 80 IRES, Aphis glycines virus 1 IRES, halastavi árva virus IRES, or pegivirus J IRES or a combination thereof.

In a preferred exemplary embodiment of the invention, the IRES is the coxsackievirus B3 IRES.

In an exemplary embodiment of the invention, the IRES is selected from SEQ ID NO: 21, SEQ ID NO: 22, SEQ ID NO: 23, SEQ ID NO: 24, SEQ ID NO:25 or SEQ ID NO: 26 or a combination thereof.

In a preferred exemplary embodiment of the invention, the IRES is sequence SEQ ID NO: 21

In an exemplary embodiment of the invention, the ORF of the ORF-coding circular mRNA is selected from the group consisting of ORF encoding virus spike protein, ORF encoding cancer antigen protein, ORF encoding reprogramming factor protein, or ORF encoding chimeric antigen receptor protein (CAR protein) or a combination thereof.

In a preferred exemplary embodiment of the invention, the ORF encoding virus spike protein is ORF encoding SARS-CoV-2 virus spike protein.

In a preferred exemplary embodiment of the invention, the ORF encoding cancer antigen protein is ORF encoding cancer antigen protein H3K27M, ORF encoding cancer antigen protein PSCA, or ORF encoding cancer antigen protein TROP2.

In a preferred exemplary embodiment of the invention, the ORF encoding reprogramming factor protein is ORF encoding reprogramming factor protein OSCK.

In a preferred exemplary embodiment of the invention, the ORF encoding chimeric antigen receptor protein (CAR protein) is ORF encoding CAR protein CD19.

### Nucleotide Sequences

In an exemplary embodiment of the invention, a T7 promoter is the T7 promoter comprising a nucleotide sequence SEQ ID NO: 1.

In an exemplary embodiment of the invention, a 5′ spacer1 is the 5′ spacer1 comprising the nucleotide sequence SEQ ID NO: 2.

In an exemplary embodiment of the invention, a 5′ external homology arm is the 5′ external homology arm comprising the nucleotide sequence SEQ ID NO: 3 or SEQ ID NO: 4.

In a preferred exemplary embodiment of the invention, the 5′ external homology arm is the nucleotide sequence SEQ ID NO: 4.

In an exemplary embodiment of the invention, an Anabaena 3′ PIE (Intron-Exon) is the Anabaena 3′ PIE comprising the nucleotide sequence SEQ ID NO: 5.

In an exemplary embodiment of the invention, a 5′ internal homology arm is the 5′ internal homology arm comprising the nucleotide sequence SEQ ID NO: 6.

In an exemplary embodiment of the invention, the 5′ spacer2 is the 5′ spacer2 comprising the nucleotide sequence SEQ ID NO: 7.

In an exemplary embodiment of the invention, an IRES according to the present invention is the IRES described in Table 1 (SEQ ID NO: 8 to 26).

TABLE 1
IRES sequences

| SEQ ID NO: | IRES |
|---|---|
| 8 | Echovirus 33 |
| 9 | Echovirus 29 |
| 10 | Echovirus 20 |
| 11 | Coxsackievirus B1 |
| 12 | Enterovirus 80 |
| 13 | Coxsackievirus A12 |
| 14 | Coxsackievirus B3 |
| 15 | Aphis glycines virus 1 |
| 16 | Halastavi árva virus 1 |
| 17 | Halastavi árva virus 2 |
| 18 | Halastavi árva virus 3 |
| 19 | Pegivirus J |
| 20 | Pegivirus J combined with Halastavi árva virus |
| 21 | Chimeric 3(20)3 |
| 22 | Chimeric 20(3)20 |
| 23 | Chimeric 3(80)3 |
| 24 | Chimeric 80(3)80 |
| 25 | Chimeric 12(20)12 |
| 26 | Chimeric 20(12)20 |

In an exemplary embodiment of the invention, an Agel restriction site is the Agel restriction site with the following nucleotide sequence:

ACCGGT

In an exemplary embodiment of the invention, an ORF according to the present invention are the ORF described in Table 2 (SEQ ID NO: 27 to 35).

TABLE 2
ORF sequences

| SEQ ID NO: | ORF |
|---|---|
| 27 | Pan-Hexapro-SPIKE spike protein |
| 28 | Pan-VFLIP-SPIKE spike protein |
| 29 | H3K27M |
| 30 | PSCA |
| 31 | TROP2 |
| 32 | OSCK |
| 33 | CAR-CD19 |
| 34 | Firefly luciferase |
| 35 | Green fluorescent protein |

In an exemplary embodiment of the invention, a NotI restriction site is the NotI restriction site with the following nucleotide sequence:

GCGGCCGC

In an exemplary embodiment of the invention, a 3′ spacer1 is the 3′ spacer1 comprising the nucleotide sequence SEQ ID NO: 36.

In an exemplary embodiment of the invention, a 3′ internal homology arm is the 3′ internal homology arm comprising the nucleotide sequence SEQ ID NO: 37.

In an exemplary embodiment of the invention, the Anabaena 5′ PIE (Intron-Exon) is the Anabaena 5′ PIE comprising the nucleotide sequence SEQ ID NO: 38.

In an exemplary embodiment of the invention, a 3′ spacer2 is the 3′ spacer2 comprising the nucleotide sequence SEQ ID NO: 39.

In an exemplary embodiment of the invention, a 3′ external homology arm is the 3′ external homology arm comprising the nucleotide sequence SEQ ID NO: 40 or SEQ ID NO: 41.

In a preferred exemplary embodiment of the invention, the 3′ external homology arm is the nucleotide sequence SEQ ID NO: 41.

In an exemplary embodiment of the invention, the plasmid vectors for making the ORF-coding circular mRNA comprising the nucleotide sequence SEQ ID NO: 42.

In an exemplary embodiment of the invention, the plasmid vector for making the ORF-coding circular mRNA, wherein an ORF is an ORF encoding the SARS-CoV-2 virus spike protein (Pan-Hexapro-SPIKE), which is a full-length comprising the nucleotide sequence SEQ ID NO: 43.

In an exemplary embodiment of the invention, the plasmid vector for making the ORF-coding circular mRNA, wherein the ORF is the ORF encoding the SARS-CoV-2 virus spike protein (Pan-VFLIP-SPIKE), which is full-length comprising the nucleotide sequence SEQ ID NO: 44.

In an exemplary embodiment of the invention, the plasmid vector for making the ORF-coding circular mRNA, wherein the ORF is the ORF encoding cancer antigen protein H3K27M, which is full-length comprising the nucleotide sequence SEQ ID NO: 45.

In an exemplary embodiment of the invention, the plasmid vector for making the ORF-coding circular mRNA, wherein the ORF is the ORF encoding cancer antigen protein PSCA comprising the nucleotide sequence SEQ ID NO: 46.

In an exemplary embodiment of the invention, the plasmid vector for making the ORF-coding circular mRNA, wherein the ORF is the ORF encoding cancer antigen protein TROP2 comprising the nucleotide sequence SEQ ID NO: 47.

In an exemplary embodiment of the invention, the plasmid vector for making the ORF-coding circular mRNA, wherein the ORF is an ORF encoding reprogramming factor protein OSCK comprising the nucleotide sequence SEQ ID NO: 48.

In an exemplary embodiment of the invention, the plasmid vector for making the ORF-coding circular mRNA, wherein the ORF is an ORF encoding chimeric antigen receptor protein CD19 comprising the nucleotide sequence SEQ ID NO: 49.

The open reading frame-coding circular mRNA (ORF-coding circular mRNA), according to this invention, has been developed at the internal ribosome entry site (IRES) to promote ribosome binding and activate the translation of proteins of interest. This results in improving protein expression by at least 2 to 10 times when compared with linear mRNA. Furthermore, it has a relatively short backbone, approximately 1,100 to 1,600 nucleotides.

For a better understanding of the invention, various examples of the circular mRNA according to the present invention will be presented. These examples are provided to illustrate embodiment of this invention and should not be construed as limiting the scope of the invention. The scope of the present invention is defined by the claims and their equivalents derived from this disclosure.

### EXAMPLES

### List of Abbreviations for Samples Used Hereinafter

“Sample A” refers to Lipofectamine® MessengerMax™ delivery system.
“Sample A/circGFP” refers to Lipofectamine® MessengerMax™ delivery system loaded with the circular GFP mRNA (CVB3 IRES) according to the invention.
“Sample A/circFluc” refers to Lipofectamine® MessengerMax™ delivery system loaded with the circular Fluc mRNA (CVB3 IRES) according to the invention.
“Sample B” refers to lipid nanoparticles formulation delivery system prepared by method according to International Journal of Pharmaceutics, 2021, 601, 120586.
“Sample B/circGFP” refers to lipid nanoparticles formulation delivery system loaded with the circular GFP mRNA (CVB3 IRES) according to the invention.
“Sample B/circFluc” refers to lipid nanoparticles formulation delivery system loaded with the circular Fluc mRNA (CVB3 IRES) according to the invention.
“Sample B/pCAG-Fluc” refers to lipid nanoparticles formulation delivery system loaded with the positive control (pCAG-Fluc DNA).
“Sample C” refers to GenVoy Ionizable Lipid Mix (GenVoy-ILM™) delivery system.
“Sample C/circGFP” refers to GenVoy Ionizable Lipid Mix (GenVoy-ILM™) delivery system loaded with the circular GFP mRNA (CVB3 IRES) according to the invention.
“Sample C1” refers to GenVoy Ionizable Lipid Mix (GenVoy-ILM™) delivery system which was formulated with a ratio between nitrogen of an ionizable lipid to phosphate of nucleic acid of 4:1 and a flow rate of about 12 ml/min.
Sample C1/circGFP” refers to GenVoy Ionizable Lipid Mix (GenVoy-ILM™) delivery system loaded with the circular GFP mRNA (CVB3 IRES) according to the invention.
“Sample C2” refers to GenVoy Ionizable Lipid Mix (GenVoy-ILM™) delivery system which was formulated with a ratio between nitrogen of an ionizable lipid to phosphate of nucleic acid of 6:1 and a flow rate of about 6 ml/min.
Sample C2/circGFP” refers to GenVoy Ionizable Lipid Mix (GenVoy-ILM™) delivery system loaded with the circular GFP mRNA (CVB3 IRES) according to the invention.
“Sample D” refers to Invivofectamine® commercial delivery system.
“Sample D/circFluc” refers to Invivofectamine® commercial delivery system loaded with the circular Fluc mRNA (CVB3 IRES) according to the invention.
“Sample E” refers to LP3000 delivery system.
“Sample E/pCAG-Fluc” refers to LP3000 delivery system loaded with the positive control (pCAG-Fluc DNA).

### Example 1 Preparation of Circular mRNA

In vitro transcription reactions were performed by the HiScribe™ T7 High Yield RNA synthesis kit using DNA templates containing ORFs and circular RNA elements. The reaction was incubated at approximately 37° C. for about 2 hours, after which the DNA templates were removed by adding deoxyribonuclease (DNase) along with the DNase buffer. The synthesized RNAs were then purified using phenol/chloroform extraction and alcohol precipitation, and the resulting RNA precipitates were dissolved in ultrapure water. The RNA concentrations were measured using a spectrophotometer, and the approximate size of RNA was determined using agarose gel electrophoresis.

For RNA circularization, RNA solutions containing nucleotide sequences of circular RNA elements were first heated at approximately 65° C. for about 5 minutes. Subsequently, they were immediately transferred onto ice and allowed to cool for about 3 minutes. The obtained RNA solutions were mixed with the guanosine-5′-triphophate (GTP) and a buffer containing Mg²⁺ ions (e.g., T4 RNA Ligase Reaction Buffer, NEB), and incubated at approximately 55° C. for about 15 minutes. The circularized products were subsequently purified by alcohol precipitation before dissolving RNA precipitates in ultrapure water. The concentration of circular RNA was measured using a spectrophotometer, and the approximate size of circular RNA was determined using agarose gel electrophoresis.

### Example 2 Study on the Efficiency of Circular mRNA Transfection into the HEK293T Cells Using Different Delivery Systems

Samples were prepared using different commercial delivery systems. HEK293T cells were transfected with approximately 1 microgram (μg) of circular mRNA (CVB3 IRES) with green fluorescent protein (GFP) (circular GFP mRNA)

### Preparation Details are as Follows:

1. Sample A/circGFP
   Sample A reagents was diluted with Opti-MEM™ medium and incubated at room temperature for about 10 minutes. The diluted circular GFP mRNA in Opti-MEM™ medium was then mixed with the prepared Sample A and incubated at room temperature for about 5 minutes to obtain the Sample A/circGFP.
2. Sample B/circGFP
   The circular GFP mRNA was mixed with Sample B using NanoAssemblr® Benchtop™ in a ratio between nitrogen of an ionizable lipid to phosphate of nucleic acid (N:P) at approximately 6:1 and a flow rate of about 6 ml/min. The prepared solution was then dialyzed with phosphate-buffered saline (PBS) to obtain the Sample B/circGFP.
3. Sample C/circGFP
   The circular GFP mRNA was mixed with Sample C using NanoAssemblr® Benchtop™ at different ratios between nitrogen of an ionizable lipid to phosphate of nucleic acid (N:P) and different flow rates. Then, the prepared solutions were dialyzed with phosphate-buffered saline (PBS) to obtained Sample C1/circGFP and sample C2/circGFP.

After 48 hours of transfection, the transfection efficiency and mean fluorescent intensity (MFI) of HEK293T cells were analyzed using flow cytometry.

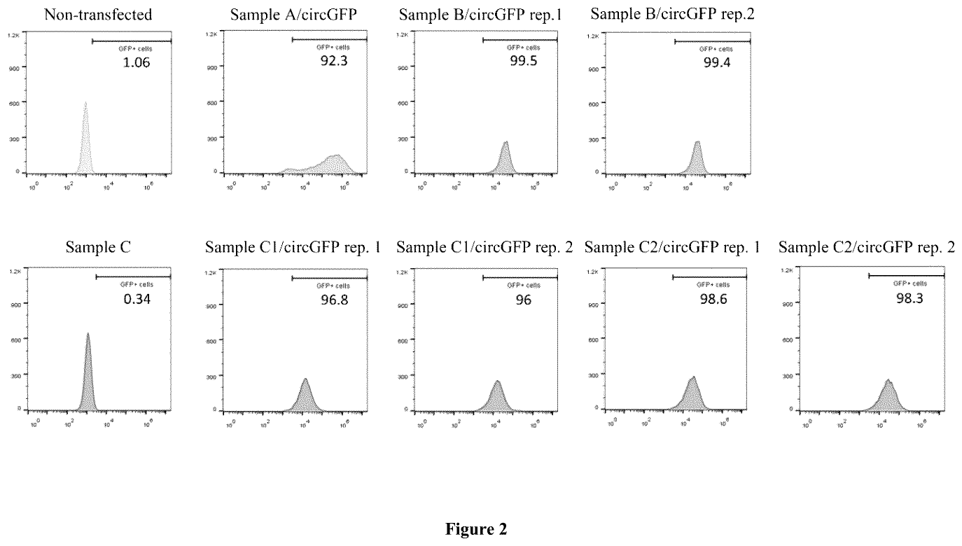

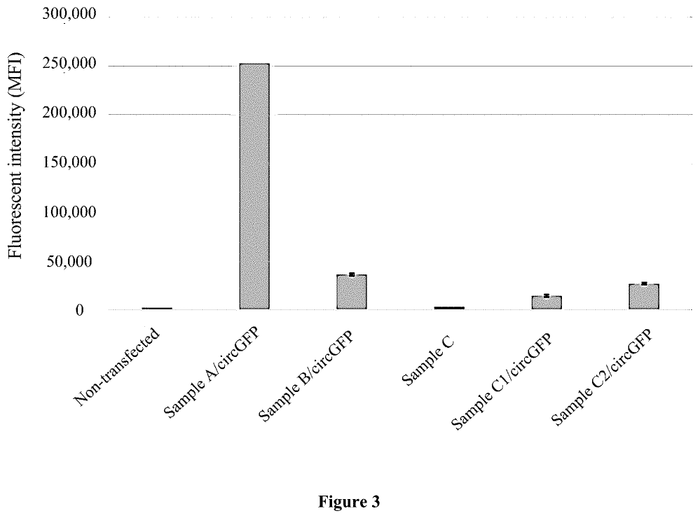

The analysis of transfection efficiency and mean fluorescent intensity demonstrated that Sample A/circGFP transfection into the HEK293T cells yielded the best efficiency and the highest expression of GFP as compared to the other samples. The transfection efficiency obtained from Sample A/circGFP was up to about 92.3%. In addition, HEK293T cells transfected with Sample A/circGFP also yielded a mean fluorescent intensity up to about 250,000 MFI as compared to the other samples as shown in FIGS. 2 and 3.

### Example 3 Study of Expression and Stability of Circular mRNA in HEK293T Cells

HEK293T cells were transfected with the circular mRNA (CVB3 IRES) that expresses luciferase (circular luciferase mRNA) of about 1 μg using different commercial delivery systems (Sample A, and D). Sample D/circFluc was prepared in the same manner of Sample A/circFluc. Following the transfection for approximately 24 and 48 hours, the transfected HEK293T cells were lysed, and the lysate cells were analyzed to measure luciferase activity using the luciferase assay.

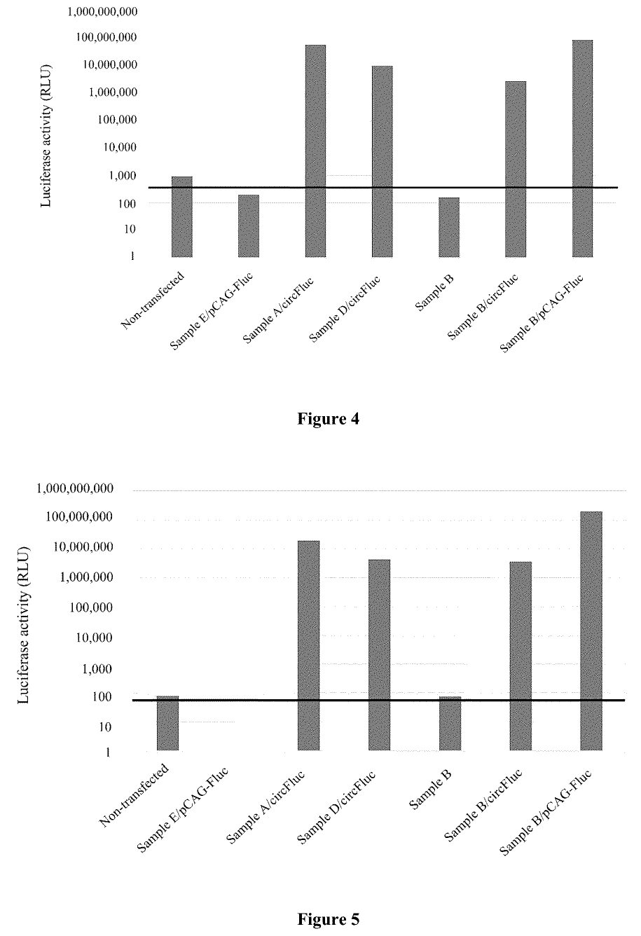

The analysis of the luciferase activity showed that Sample A/circFluc, and Sample D/circFluc yielded higher luciferase expression compared to the transfected positive control which are Sample B/pCAG-Fluc and Sample E/pCAG-Fluc. Sample E was prepared in the same manner of sample A. Sample A/circFluc and Sample D/circFluc yielded the luciferase expression, ranging from about 10,000,000 to 100,000,000 RLU (relative light units) and ranging from about 1,000,000 to 100,000,000 RLU at about 24 hours and about 48 hours, as shown in FIG. 4 and FIG. 5, respectively. In addition, after the transfection, the analysis of luciferase activity also demonstrated that the Sample A/circFluc and Sample D/circFluc remained stable in HEK293T cells for up to 48 hours, with no significant difference in luciferase activity the 24-hour and 48-hour time points, as shown in FIGS. 4 and 5.

### Example 4 Study of Expression and Stability of Circular mRNA with Various Kinds of IRES in HEK293T Cells

Each of IRES sequences, i.e., coxsackievirus B3 IRES (CVB3) (SEQ ID NO: 14), Aphis glycines virus 1 IRES (A51) (SEQ ID NO: 15), halastavi árva virus IRES (H5I, HCl, HII) (SEQ ID NO: 16, 17, 18, respectively), pegivirus J IRES (Peg) (SEQ ID NO: 19), and a combination of pegivirus J and halastavi árva virus IRES (CPH) (SEQ ID NO: 20), was cloned into a circular GFP vector. These circular GFP vectors were used to make circular mRNA. Subsequently, about 2 μg of the circular mRNA was transfected into HEK293T cells using the Sample A as a delivery system. Following transfection at approximately 48 hours, the HEK293T cells were analyzed using flow cytometry.

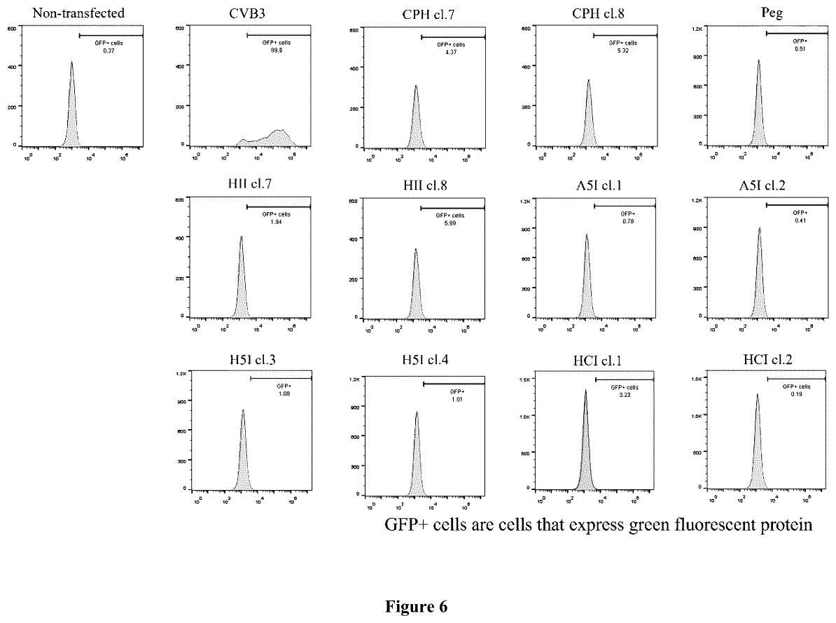

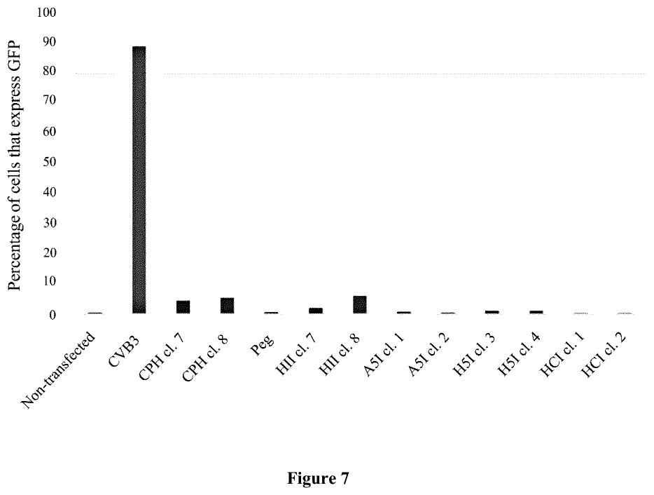

The analysis of GFP expression and stability of circular mRNA with different IRES in the HEK293T cells showed that the circular mRNA with CVB3 IRES yielded the highest expression with positive results, up to about 89%, as compared to the cells that were transfected with the circular mRNA with the other IRES. In addition, such flow cytometer results also demonstrated that circular mRNA with CVB3 IRES was also stable in HEK293T cells for about 48 hours, as shown in FIGS. 6 and 7.

### Example 5 Study of Expression and Stability of Circular mRNA in Animals

Mice (BALB/c species, approximately 7 weeks old, female) were administered with the Sample B/circFluc via intramuscular (I.M.) and intravenous (I.V.) routes at doses of about 1 μg and about 10 μg. At approximately 24 and 48 hours post-injection, the mice were anesthetized, and their luminescent values were measured using the IVIS® spectrum in vivo imaging system.

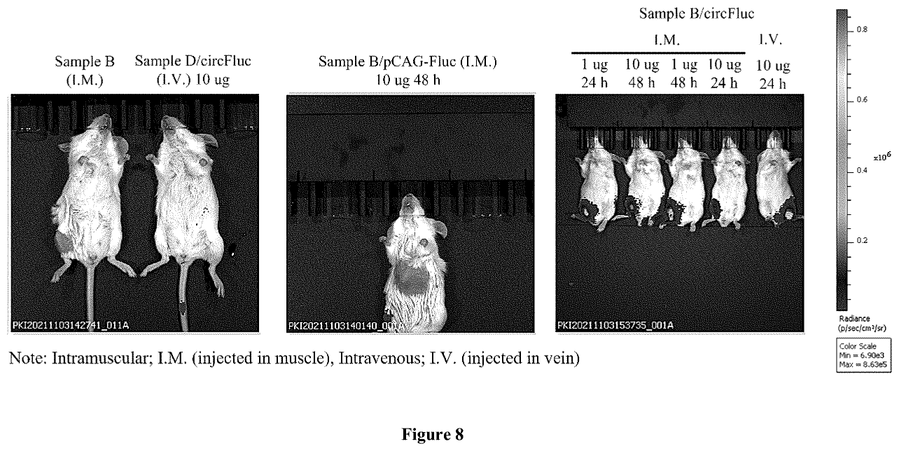

The analysis of the luminescent image demonstrated that the Sample B/circFluc, has the highest expression, and long stability in mice for about 48 hours, regardless of its injection routes (intramuscular or intravenous injection) or doses (about 1 μg or about 10 μg), as compared to the positive controls (Sample B/pCAG-Fluc), at the same conditions (FIG. 8).

### Example 6 Study of Expression and Stability of Circular SARS-CoV-2 Spike Protein mRNA in HEK293T Cells

Human Embryonic Kidney 293T (HEK293T) cells were transfected with the circular mRNA (CVB3 IRES) expressing SARS-CoV-2 spike protein in two forms: Pan-Hexapro-SPIKE (HexaPro-VI) and Pan-VFLIP-SPIKE (VFLIP-VI) at a dose of approximately 1 μg. The positive control was SARS-CoV-2 spike protein DNA (pCMV3-S DNA) at about 1 μg.

Following transfection at approximately 48 hours, HEK293T cells were stained with the SARS-CoV-2 (2019-nCOV) Spike RBD Antibody (Rabbit PAb, Antigen Affinity Purified (40592-T62, SinoBiological)) and the Goat Anti-Rabbit IgG (H+L) Highly Cross-Adsorbed Secondary Antibody (Alexa Fluor 488 (A11034, Invitrogen)). The efficiency of expression was then analyzed using flow cytometry.

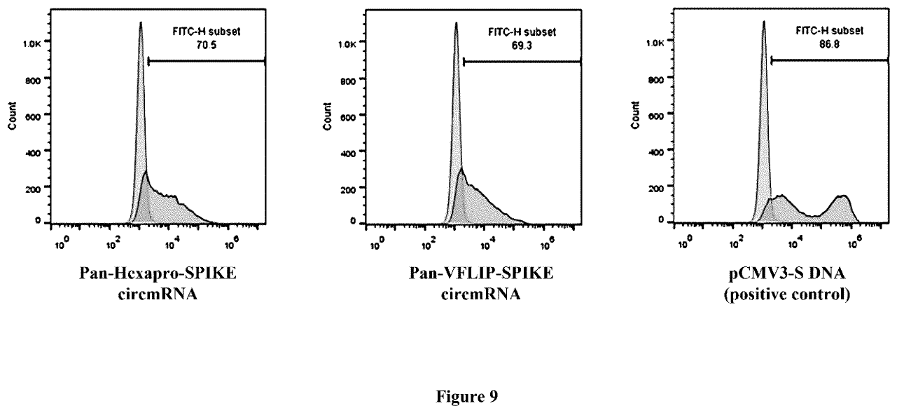

The analysis of flow cytometry demonstrated that the circular SARS-CoV-2 spike protein mRNA (CVB3 IRES), in both Pan-Hexapro-SPIKE and Pan-VFLIP-SPIKE forms, yielded high expression of spike proteins, as compared to positive controls (pCMV3-S DNA) (FIG. 9).

In addition, HEK293T cells were transfected with circular SARS-CoV-2 spike protein mRNAs (CVB3 IRES) which are Pan-Hexapro-SPIKE (HexaPro-VI) and Pan-VFLIP-SPIKE (VFLIP-VI). The circular SARS-CoV-2 spike protein mRNAs were lysed, and the lysate cells were analyzed by western blot to determine the expression of SARS-CoV-2 spike protein in the supernatant. The cells were stained with SARS-CoV-2 spike RBD Antibody (Rabbit PAb), (Affinity Purified (40592-T62, SinoBiological)) and donkey anti-rabbit IgG-HRP (sc-2077, SANTA CRUZ BIOTECHNOLOGY, INC.). The internal control protein was β-Actin protein.

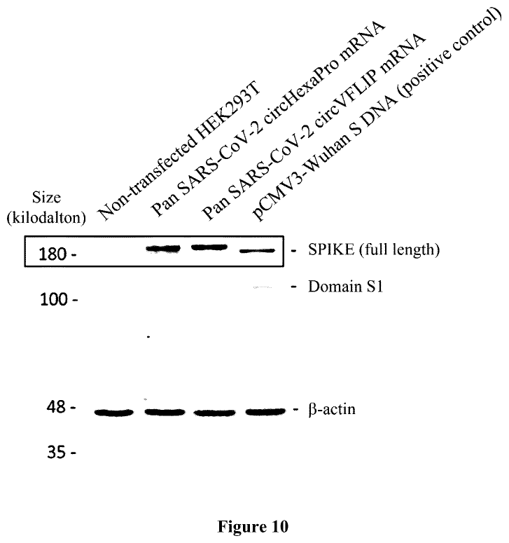

From the western blot, it was found that both Pan-Hexapro-SPIKE and Pan-VFLIP-SPIKE forms of the circular SARS-CoV-2 spike protein mRNA (CVB3 IRES) yielded high expression of spike proteins compared to positive controls (pCMV3-S DNA), as shown in FIG. 10.

### Example 7 Study of Expression and Stability of Circular SARS-CoV-2 Spike Protein mRNA in Experimental Animals

Mice (BALB/c species, approximately 7 weeks old, female) were injected with approximately 5 μg of the circular mRNA (CVB3 IRES) expressing SARS-CoV-2 spike proteins of Pan-Hexapro-SPIKE (HexaPro-VI) and Pan-VFLIP-SPIKE (VFLIP-VI) using the Sample B.

The administered route involved two intramuscular (I.M.) injections, with the second dose given three weeks after the first. Two weeks after the second injection, the serum samples from the mice were analyzed using enzyme-linked immunosorbent assay (ELISA) to determine the expression level of immunoglobulin G (IgG) specific to the SARS-CoV-2 spike protein Omicron variant (B.1.1.529), in comparison to a control group of mice that received phosphate-buffered saline (PBS) injections.

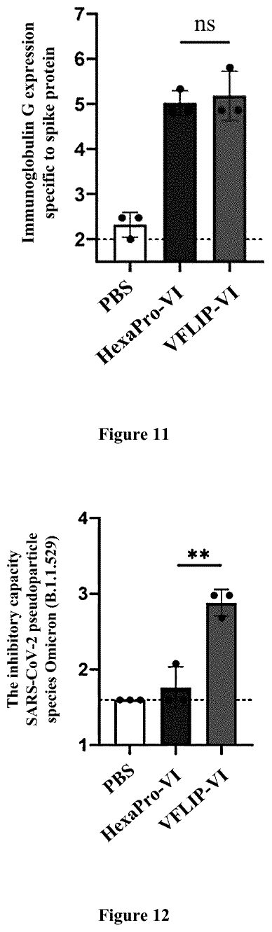

The ELISA results indicated that both forms of circular SARS-CoV-2 spike protein mRNA (CVB3 IRES), Pan-Hexapro-SPIKE (HexaPro-VI) and Pan-VFLIP-SPIKE (VFLIP-VI), were able to elicit expression of the IgG specific to SARS-CoV-2 spike protein Omicron species (B.1.1.529) in mice serum, as compared to the control group of mice (as shown in FIG. 11).

In addition, the serum samples obtained from mice that received both forms of circular SARS-CoV-2 spike protein mRNA, Pan-Hexapro-SPIKE (HexaPro-VI) and Pan-VFLIP-SPIKE (VFLIP-VI), were examined to determine the titers of antibodies specific to SARS-CoV-2 pseudovirus spike protein Omicron species (B.1.1.529) using a neutralization assay. This analysis was conducted to compare the results with those obtained from the control group of mice that received injections of phosphate-buffered saline (PBS).

The analysis of the neutralization assay revealed that serum obtained from the mice that received injections of the circular mRNA (CVB3 IRES) expressing Pan-VFLIP-SPIKE (VFLIP-VI) spike protein exhibited the production of antibodies specific to SARS-CoV-2 pseudovirus spike protein Omicron species (B.1.1.529), in comparison to the control group of mice (as illustrated in FIG. 12).

### Example 8 Study of Expression and Stability of Circular Cancer Antigen Protein mRNA in HEK293T Cells

HEK293T cells were transfected with approximately 2 μg of the circular cancer antigen protein mRNAs including H3K27M, PSCA, or TROP2.

Following the transfection at approximately 48 hours, HEK293T cells were analyzed for protein expression using immunofluorescence assay. The cells were subjected to staining with recombinant anti-histone H3 (mutated K27M) antibody ([EPR18340]-ChIP Grade (ab190631)) and goat anti-rabbit IgG (H+L) highly cross-adsorbed secondary antibody (Alexa Fluor 594 (A-11037, Thermo Fisher)) to detect the H3K27M protein expression. Anti-PSCA antibody (rabbit polyclonal (201684-T32, SinoBiological)) and the goat anti-rabbit IgG (H+L) highly cross-adsorbed secondary antibody (Alexa Fluor 594 (A-11037, Thermo Fisher)) were used to stain for the PSCA protein expression. Moreover, recombinant anti-TROP2 antibody (APC) (rabbit monoclonal (10428-R001-A, SinoBiological)) was used to stain for the TROP2 protein expression.

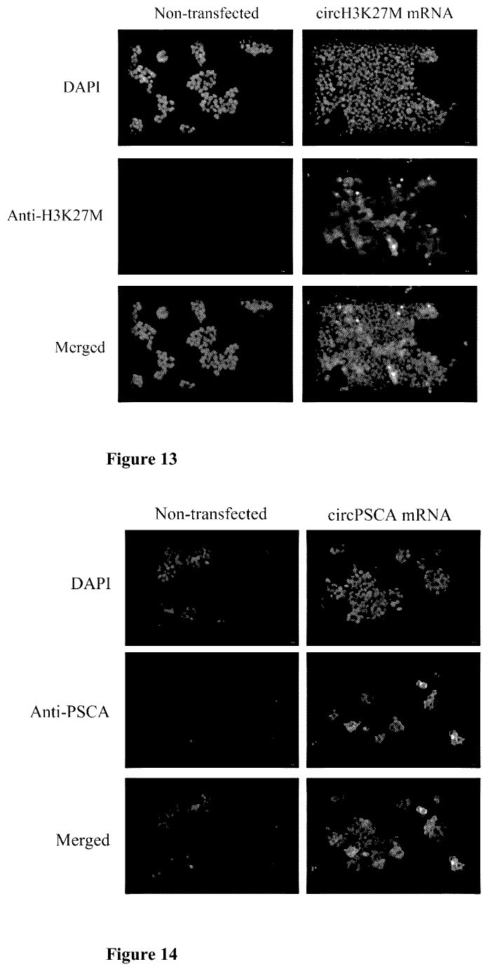

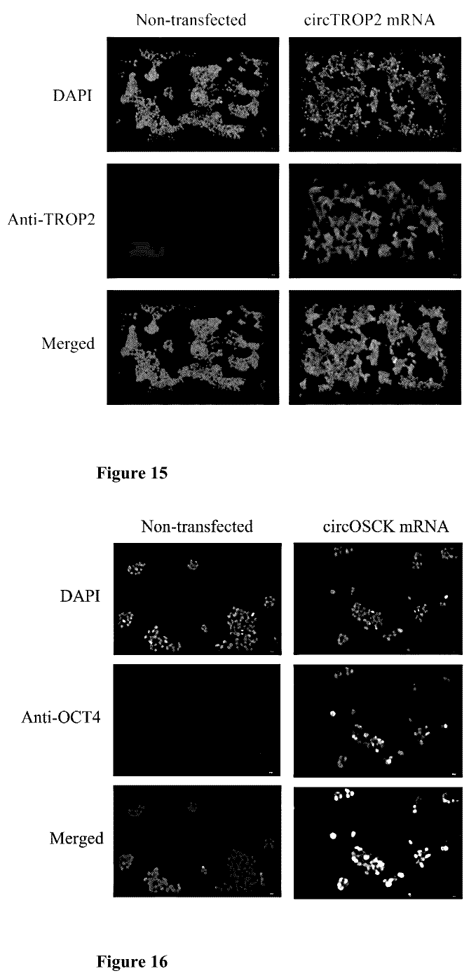

The analysis of the immunofluorescence assay showed that the circular cancer antigen protein mRNA in all three of its forms (i.e., H3K27M, PSCA, and TROP2), led to the expression of corresponding cancer antigen protein. This was evident from the red staining observed in the cells, which was comparable to the expression of cancer antigen proteins in non-transfected cells (as depicted in FIGS. 13 to 15).

### Example 9 Study of Expression and Stability of Circular Reprogramming Factor mRNA in HEK293T Cells

HEK293T cells were transfected with approximately 2 μg of the circular reprogramming factor mRNA OSCK.

Following the transfection at approximately 48 hours, HEK293T cells were analyzed for protein expression using immunofluorescence assay. The cells were stained with anti-oct-3/4 antibody ((C-10) (sc-5279, Santa Cruz)) and goat anti-mouse IgG (H+L) highly cross-adsorbed secondary antibody (Alexa Fluor 488 (A-11290, Thermo Fisher)) to detect OCT4 protein, Sox2 (D6D9) XP® rabbit mAb (3579, Cell Signaling Technology) and the goat anti-mouse IgG (H+L) highly cross-adsorbed secondary antibody (Alexa Fluor 488 (A-11290, Thermo Fisher)) were used to stain for SOX2 protein expression.

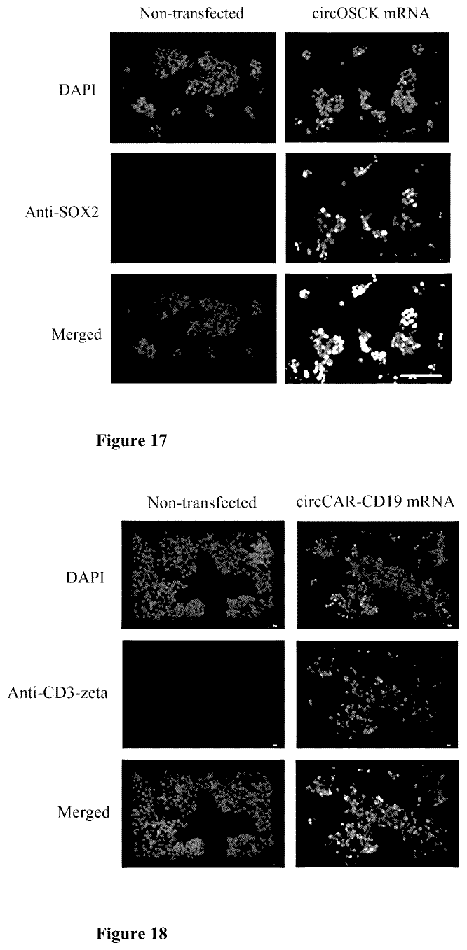

The analysis of the immunofluorescence assay showed that the circular reprogramming factor mRNA OSCK led to the expression of reprogramming factor protein. This was evident from the green staining observed in the cells, which was comparable to the expression of cancer antigen proteins in non-transfected cells (as depicted in FIGS. 16 to 17).

### Example 10 Study of Expression and Stability of Circular Chimeric Antigen Receptor mRNA (Circular CAR mRNA) in HEK293T Cells

HEK293T cells were transfected with approximately 2 μg of the circular mRNA (CVB3 IRES) expressing chimeric antigen receptor (circular CAR mRNA) CD19.

After transfection at approximately 48 hours, HEK293T cells were analyzed for protein expression using immunofluorescence assay. The cells were subjected to staining with CD3-zeta polyclonal antibody (PA5-98304, Thermo Fisher) and goat anti-mouse IgG (H+L) highly cross-adsorbed secondary antibody (Alexa Fluor 488 (A-11290, Thermo Fisher)) to detect CD19 protein.

The analysis of the immunofluorescence assay showed that the circular CAR mRNA (CVB3 IRES) CD19 led to the expression of chimeric antigen receptor protein. This was evident from the red staining observed in the cells, which was comparable to the expression of chimeric antigen receptor proteins in the non-transfected cells (as depicted in FIG. 18).

### Example 11 Circular RNA Preparation

To further prepare circular RNA using various types of IRES, DNA templates were designed to include regions for circular ribonucleic acid. Polymerase chain reaction (PCR) was carried out to obtain DNA precursor for in vitro RNA production. All single-stranded RNAs were synthesized using the HiScribe™ T7 High Yield RNA synthesis kit from New England Biolabs (NEB). A 10 μl of reaction mixture was prepared with a final 1× IVT reaction buffer, 10 mM NTPs (ATP, UTP, CTP, and GTP), 500 ng of DNA precursor, RNase inhibitor, and T7 RNA polymerase mix. The reaction mixture was then incubated at approximately 37° C. for approximately 2 hours. The DNA precursor was subsequently removed by the addition of DNase I, and the mixture was incubated at about 37° C. for approximately 15 minutes. The obtained RNA were purified using the Monarch RNA column kit (NEB). Finally, RNA gel electrophoresis was performed to verify RNA integrity.

To circularize the RNA, 50 μg of linear RNA from the in vitro transcription (IVT) reaction was heated at about 65° C. for about 5 minutes. Subsequently, it was immediately transferred onto ice and allowed to cool for about 1 minute. Next, the obtained RNA was added into a reaction containing 2 mM GTP and Mg²⁺ in a 1× T7 RNA ligase buffer (NEB) and circularized at about 55° C. for about 15 minutes. The reaction was immediately by transferring the solution onto ice. The circularized products (IC) were subsequently purified using the Monarch RNA column kit (NEB), and the approximate size of the circular RNA was determined by agarose gel electrophoresis. The resulting circularized IVT product is referred to as IC.

The species of the obtained RNA from IC products was verified. Briefly, the sample was treated with exonuclease RNase R, which specifically digests linear RNA, at about 37° C. for about 2 hours. The entire reaction was subsequently purified using the Monarch RNA column kit (NEB). The approximate size of circular RNA was determined using agarose gel electrophoresis.

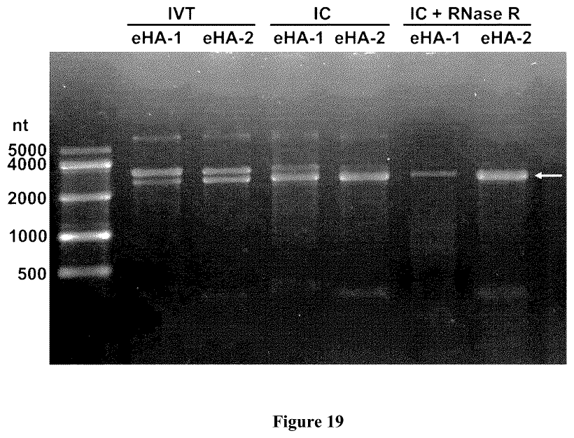

The circular RNA-eHA-1 and eHA-2 bands, representing the circular RNA obtained after treatment with RNase R, were visible in the IC and RNase R lanes, as depicted in FIG. 19. The homology arm eHA-2 duplex is designed to possess higher free energy than the homology arm eHA-1 duplex.

### Example 12 Study of Expression and Stability of Circular mRNA in HEK293T Cells

HEK293T cells were plated at a density of about 50,000 cells/cm² and incubated at about 37° C. in 5% CO₂. After 24 hours of culture in Dulbecco's Modified Eagle's Medium (DMEM) high glucose (Cytiva) supplemented with 10% heat-inactivated fetal bovine serum (Sigma-Aldrich), the cells were ready for RNA transfection. Circular RNAs with different designs of 5′ external homology arms (eHA) (eHA-1 or eHA-2) expressing firefly luciferase (Fluc) were added into cells using Lipofectamine® MessengerMax™ (Invitrogen). The cells were washed once with 1×PBS and harvested at 48 hours after transfection. The cells were counted and calculated to be 500,000 cells per reaction. To perform cell lysis, 1× cell culture lysis reagent was introduced. Luciferase assay reagent was added into a luminometer tube followed by cell lysate in a 5:1 volume ratio of luciferase assay reagent to cell lysate. Then, luciferase activity was monitored using a luminometer.

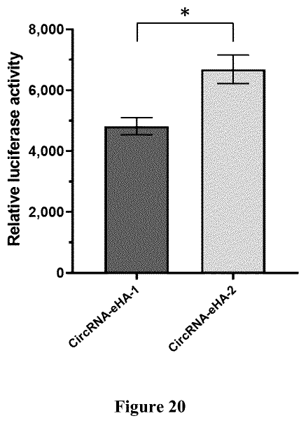

The analysis of luciferase activity between two different designed constructs showed that circular luciferase mRNA with eHA-2 expressed higher luciferase activity than eHA-1 construct. At 24 hours post-transfection, the circular luciferase mRNA yielded the luciferase expression up to about 5,000 to 7,000 RLU (relative light units) (see FIG. 20). The results of gel electrophoresis (FIG. 19) and the luciferase activity measurements (FIG. 20) both showed that the circular RNA-eHA-2 construct had higher circularization efficiency and luciferase activity of than circular RNA-eHA-1 construct. Based on these findings, the eHA-2 design of the external homology arm was used to construct circular RNAs, and CVB3 IRES was replaced with other types of IRES, including natural or chimeric sequences.

### Example 13 Study of Expression and Stability of Circular mRNA with Various Kinds of IRES in the imHC and BHK-21 Cells

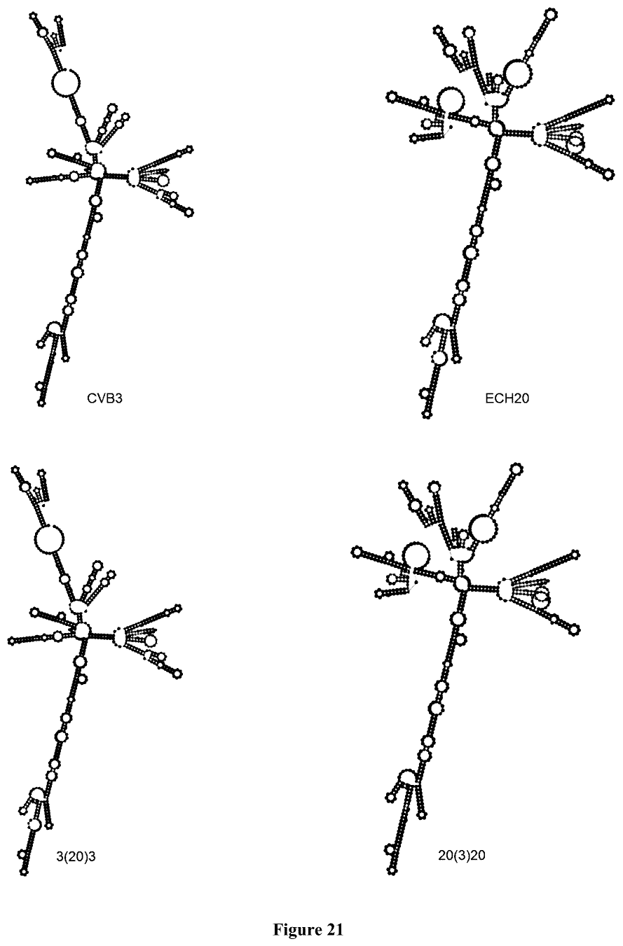

The chimeric sequences were engineered using domain IV substitution. For example, when domain IV of CVB3 IRES was substituted with that of ECH20 IRES, the resulting chimeric IRES is designated 3 (20) 3 (FIG. 21). The CircRNA-eHA-2 engineered using the chimeric IRES 3 (20) 3 is designated 3 (20) 3 in figures where relevant. Hepatocyte-like cells that were derived from mesenchymal stem cells (imHC) or baby hamster kidney fibroblast (BHK-21) cells were plated at a density of approximately 50,000 cells/cm² and incubated at about 37° C. in 5% CO₂. After 24 hours of culture in Dulbecco's Modified Eagle's Medium (DMEM) high glucose (Cytiva) supplemented with 10% heat-inactivated fetal bovine serum (Sigma-Aldrich) for about 24 hours, the cells were ready for RNA transfection. Various kinds of IRES sequences including chimeric IRES 3 (20) 3 (SEQ ID NO: 21), chimeric IRES 20 (3) 20 (SEQ ID NO: 22), chimeric IRES 3 (80) 3 (SEQ ID NO: 23), chimeric IRES 80 (3) 80 (SEQ ID NO: 24), chimeric IRES 12 (20) 12 (SEQ ID NO: 25), and chimeric IRES 20 (12) 20 (SEQ ID NO: 26) were used. Circular RNA-eHA-2 constructs with different IRES expressing green fluorescent protein (GFP) were added into cells using Lipofectamine® MessengerMax™ (Invitrogen). After 24 hours of transfection, GFP fluorescent signals were visualized using the fluorescent microscope.

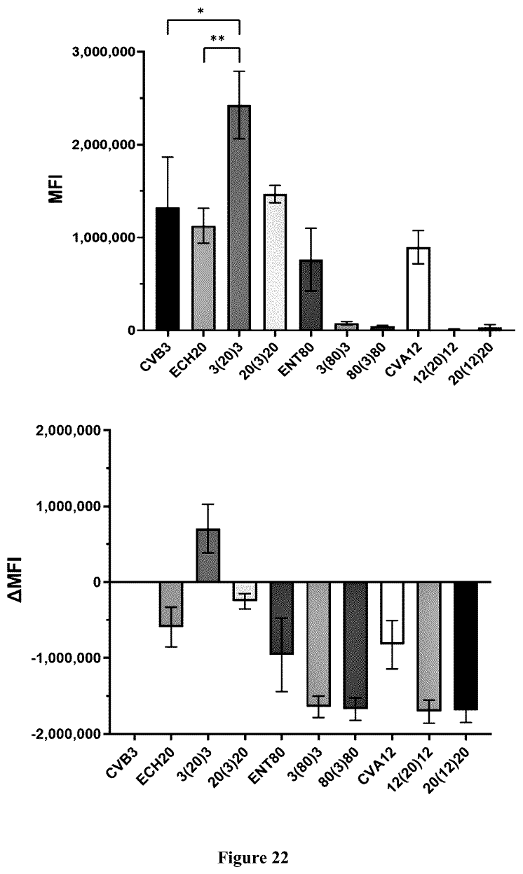

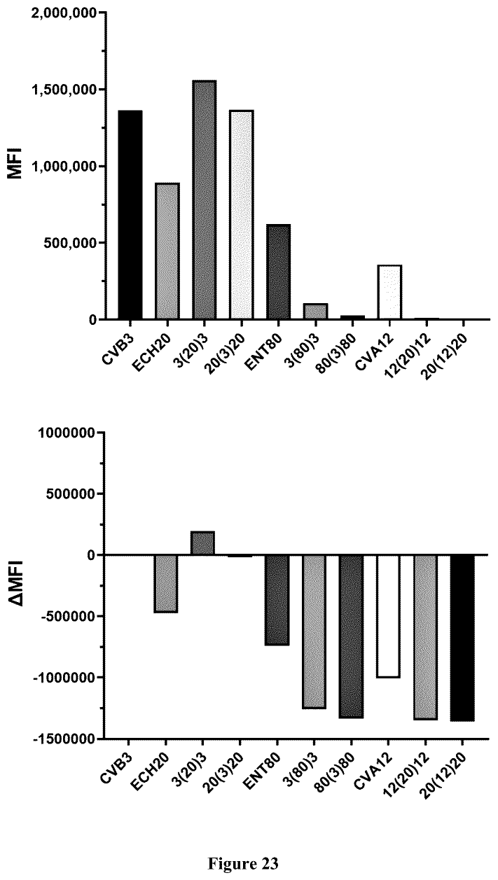

The analysis of fluorescence intensity revealed that circular luciferase mRNA, using the different IRES, showed different GFP expressions in the imHC and BHK-21 cells. Specifically, the circular luciferase mRNA with 3 (20) 3 IRES had the highest mean fluorescence intensity (MFI) and different mean fluorescence intensity (ΔMFI) of GFP compared to other IRES types (see FIGS. 22 and 23).

### Example 14 Study of Expression and Stability of Circular mRNA with Different IRES in Experimental Animals

In this study, Moderna Inc's formula was used as a guide to select lipid components for the construction of lipid nanoparticles (LNP) for encapsulating circular RNA. Circular RNA-LNP encapsulation was performed using the NanoAssemblr® Benchtop microfluidic device (Precision Nanosystems). To briefly summarize the process, lipid containing ethanol phase and circular RNA containing aqueous phase were mixed at the volumetric ratio of 1:3. The ethanol phase comprised the mixture of SM-102, DSPC, cholesterol and DMG-PEG 2000 in absolute ethanol, all of which were obtained from Sinopeg. The aqueous phase was circular RNA in 25 mM acetate buffer at pH 4.0. After encapsulation, the concentration of the circular RNA-LNP was investigated using the Qubit™ RNA high sensitivity assay kit (Thermo Fisher). The functionality of the circular RNA-LNP was verified by assessing in vitro luciferase activity before administration to mice.

For intramuscular (I.M.) injection in mice, firstly, circular FLuc-eHA-2-CVB3 or circular FLuc-eHA-2-3 (20) 3 encapsulated in LNP at an amount of 5 μg was injected into the right hind limb of female BALB/c mice (n=3) using an insulin syringe. As a negative control, PBS was administered. Mice were anesthetized with 2% isoflurane inhalation in an airtight transparent anesthesia box for 3-5 minutes at 6 or 48 hours after administration. After anesthesia, the mice were intraperitoneally injected with D-luciferin (150 micrograms per kilogram (mg/kg) in 200 microliters (μl) of PBS) and placed into the imaging chamber of an in vivo imaging system (IVIS) for imaging. Bioluminescent imaging (BLI) was performed using the IVIS spectrum, and sequential images were acquired at 1-minute intervals with 60-second exposure time.

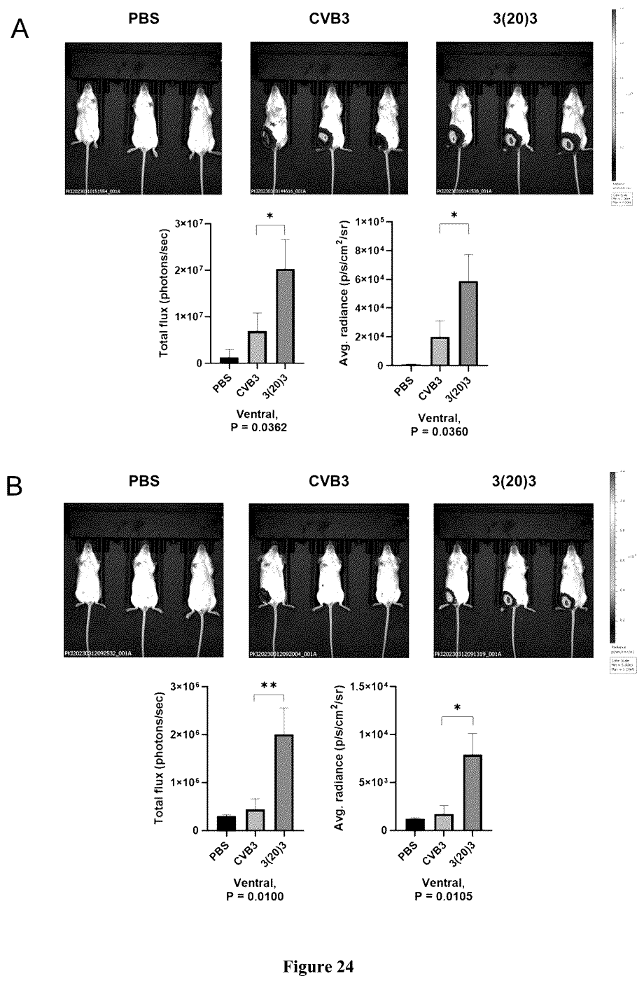

The analysis of the bioluminescent imaging showed that the circular luciferase mRNA with eHA-2-3 (20) 3 exhibited the highest expression and maintained stability in mice at both 6 and 48 hours approximately after intramuscular (I.M.) injection, as compared to the negative control (PBS) (see FIG. 24).

### BEST MODE OF THE INVENTION

Best mode of the invention is as provided in the description of the invention.

1. An internal ribosome entry site (IRES) comprising a nucleotide sequence selected from the group consisting of SEQ ID NO: 21, SEQ ID NO: 22 or a combination thereof, preferably a nucleotide sequence as shown in SEQ ID NO: 21.

2. A plasmid vector for making an open reading frame-coding circular mRNA, said plasmid vector comprising elements connected to each other and arranged in a following sequence:
RNA polymerase promoter,
5′ spacer1,
5′ external homology arm,
3′ PIE (permuted intron-exon),
5′ internal homology arm,
5′ spacer2,
Internal ribosome entry site (IRES),
Open reading frame (ORF),
3′ spacer1,
3′ internal homology arm,
5′ PIE (permuted intron-exon),
3′ spacer2, and
3′ external homology arm
wherein the IRES is selected from the group consisting of SEQ ID NO: 21, SEQ ID NO: 22 or a combination thereof.

3. The plasmid vector according to claim 2, wherein the RNA polymerase promoter has a length from 15 to 25 nucleotides, preferably selected from the group consisting of T7 virus RNA polymerase promoter, SP6 virus RNA polymerase promoter, or T3 virus RNA polymerase, more preferably the T7 virus RNA polymerase promoter.

4. The plasmid vector according to claim 2, wherein the 5′ spacer1 has a length from 5 to 15 nucleotides.

5. The plasmid vector according to claim 2, wherein the 5′ external homology arm has a length from 15 to 30 nucleotides, preferably from 15 to 20 nucleotides or 20 to 30 nucleotides, more preferably sequence SEQ ID NO: 4.

6. The plasmid vector according to claim 2, wherein the 3′ PIE (permuted intron-exon) has a length from 100 to 250 nucleotides, preferably obtained from Cyanobacterium anabaena pre-tRNA group I intron gene.

7. The plasmid vector according to claim 2, wherein the 5′ internal homology arm has a length from 15 to 25 nucleotides.

8. The plasmid vector according to claim 2, wherein the 5′ spacer2 has a length from 50 to 100 nucleotides.

9. The plasmid vector according to claim 2, wherein the IRES has a length from 190 to 900 nucleotides, preferably sequence SEQ ID NO: 21.

10. The plasmid vector according to claim 2, wherein the ORF is selected from the group consisting of:
ORF encoding virus spike protein, wherein the ORF encoding virus spike protein is ORF encoding SARS-CoV-2 virus spike protein;
ORF encoding cancer antigen protein, wherein the ORF encoding cancer antigen protein is selected from ORF encoding cancer antigen protein H3K27M, ORF encoding cancer antigen protein PSCA, or ORF encoding cancer antigen protein TROP2;
ORF encoding reprogramming factor protein, wherein the ORF encoding reprogramming factor protein is ORF encoding reprogramming factor protein OSCK;
ORF encoding chimeric antigen receptor protein (CAR protein), wherein the ORF encoding chimeric antigen receptor protein (CAR protein) is ORF encoding CAR protein CD19; or
a combination thereof.

11. The plasmid vector according to claim 2, wherein the 3′ spacer1 has a length from 15 to 25 nucleotides.

12. The plasmid vector according to claim 2, wherein the 3′ internal homology arm has a length from 15 to 25 nucleotides.

13. The plasmid vector according to claim 2, wherein the 5′ PIE (permuted intron-exon) has a length from 100 to 250 nucleotides, preferably obtained from Cyanobacterium anabaena pre-tRNA group I intron gene.

14. The plasmid vector according to claim 2, wherein the 3′ spacer2 has a length from 5 to 15 nucleotides.

15. The plasmid vector according to claim 2, wherein the 3′ external homology arm has a length from 15 to 30 nucleotides, preferably from 15 to 25 nucleotides or 25 to 30 nucleotides, more preferably sequence SEQ ID NO: 41.

16. ORF-coding circular mRNA obtained from the plasmid vector according to claim 2.

17. An open reading frame-coding circular mRNA (ORF-coding circular mRNA) obtained from a plasmid vector comprising elements that are connected to each other and arranged in a following sequence:
3′ exon
Internal ribosome entry site (IRES)
Open reading frame (ORF)
5′ exon
wherein the IRES is selected from the group consisting of SEQ ID NO: 21, SEQ ID NO: 22 or a combination thereof.

18. The ORF-coding circular mRNA according to claim 17, wherein the IRES has a length from 190 to 900 nucleotides, preferably sequence SEQ ID NO: 21.

19. The ORF-coding circular mRNA according to claim 17, wherein the ORF is selected from the group consisting of:
ORF encoding virus spike protein, wherein the ORF encoding virus spike protein is ORF encoding SARS-CoV-2 virus spike protein;
ORF encoding cancer antigen protein, wherein the ORF encoding cancer antigen protein is selected from ORF encoding cancer antigen protein H3K27M, ORF encoding cancer antigen protein PSCA, or ORF encoding cancer antigen protein TROP2;
ORF encoding reprogramming factor protein, wherein the ORF encoding reprogramming factor protein is ORF encoding reprogramming factor protein OSCK;
ORF encoding chimeric antigen receptor protein (CAR protein), wherein the ORF encoding chimeric antigen receptor protein (CAR protein) is ORF encoding CAR protein CD19; or
a combination thereof.

20.-40. (canceled)


SEQUENCE LISTING (49 sequences)

SEQ ID NO: 1 - DNA, 19 bp, synthetic construct
  source 1..19: mol_type = other DNA
taatacgactcactatagg

SEQ ID NO: 2 - DNA, 11 bp, synthetic construct
  source 1..11: mol_type = other DNA
gagaccctcga

SEQ ID NO: 3 - DNA, 21 bp, synthetic construct
  source 1..21: mol_type = other DNA
gattggatacaaacacggctc

SEQ ID NO: 4 - DNA, 27 bp, synthetic construct
  source 1..27: mol_type = other DNA
ccagggatcccagcaatgagaatccta

SEQ ID NO: 5 - DNA, 182 bp, synthetic construct
  source 1..182: mol_type = other DNA
aacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccaccccca

SEQ ID NO: 6 - DNA, 20 bp, synthetic construct
  source 1..20: mol_type = other DNA
cgccggaaacgcaatagccg

SEQ ID NO: 7 - DNA, 65 bp, synthetic construct
  source 1..65: mol_type = other DNA
ggcccaaaaacaaaaaacaaaaaaaacaaaaaaaaaaccaaaaaaacaaaacacagcatgccaca

SEQ ID NO: 8 - DNA, 737 bp, synthetic construct
  source 1..737: mol_type = other DNA
ttaaaacagcctgtgggttgatcccacccacagggcccattgggcgctagcactctggtatcacggtacccttgtgcgcctgttttatgtcccttccctcaactgtaacttagaagtaacgcacaccgatcaacagtcagcgtggcacaccagccatgttttgatcaagcacttctgttaccccggaccgagtatcaacagactgctcacgcggttgaaggagaaagtgttcgttatccggccaactacttcgaaaaacctagtaacaccatggaagttgcagagtgtttcgctcagcactaccccagtgtagatcaggtcgatgagtcaccgcatcccccacgggcgaccgtggcggtggctgcgttggcggcctgcctatgggggaacccataggacgctctaatacagacatggtgcgaagagtccattgagctagttggtagtcctccggcccctgaatgcggctaatcctaactgcggagcacacaccttcaagccagagggcagtgtgtcgtaacgggcaactctgcagcggaaccgactactttgggtgtccgtgtttcattttattcttatactggctgcttatggtgacaattgagagattgttaccatatagctattggattggccatccagtgactagcagagctattatatacctctttgttgggtttataccacctaatttgaaagaagttaaaacattagaattcattattaaattgaataca

SEQ ID NO: 9 - DNA, 742 bp, synthetic construct
  source 1..742: mol_type = other DNA
ttaaaacagcctgtgggttgatcccacccacagggcccactgggcgctagcactctggtatcacggtacctttgtgcgcctgttttatacttcctcccccaactgcaacttagaagtaacacaaaccgatcaacagtcagcgtggcacaccagccacgttttgatcaaacacttctgttaccccggactgagtatcaatagactgctcacgcggttgaaggagaaaacgttcgttatccggccaactacttcgagaaacctagtaacgccatggaagttgtggagtgtttcgctcagcactaccccagtgtagatcaggttgatgagtcaccgcattccccacgggtgaccgtggcggtggctgcgttggcggcctgcccatggggaaacccatgggacgctcttatacagacatggtgcgaagagtctattgagctagttggtagtcctccggcccctgaatgcggctaatcccaactgcggagcatacactctcaagccagagggtagtgtgtcgtaatgggcaactctgcagcggaaccgactactttgggtgtccgtgtttcattttattcctatactggctgcttatggtgacaattgagagattgttaccatatagctattggattggccatccggtgactaacagagctattatatatctttttgttgggtttataccacttagcttgaaagaggttaaaactctacattacattttaatactgaacaccgcaaa

SEQ ID NO: 10 - DNA, 742 bp, synthetic construct
  source 1..742: mol_type = other DNA
ttaaaacagcctgtgggttgttcccacccacagggcccattgggcgctagcactctggtattacggtacctttgtgcgcctgttttatgtcccctcccccaatcgcaacttagaagcaacacacactgatcaacagtaagcgtggcataccagccacgttttgatcaagcacttctgttaccccggactgagtatcaatagactgctcacgcggttgaaggagaaagcgttcgttatccggccaactacttcgagaaacctagtaacaccgtgaaagttgcagagtgtttcgctcagcactaccccagtgtagatcaggtcgatgagtcaccgcattccccacgggcgaccgtggcggtggctgcgctggcggcctgcctacggggaaacccgtaggacgctctaatacagacatggtgcgaagagtctattgagctagttggtagtcctccggcccctgaatgcggctaatcctaactgcggagcacacacccccaagccagggggcagtgtgtcgtaacgggtaactctgcagcggaaccgactactttgggtgtccgtgtttcattttattcctatgctggctgcttatggtgacaattgacagattgttaccatatagctattggattggccatccggtgactaatagagccattatataccactttgttgggtttataccactcaacttgaaagaggtcaaaacactacagctcatcattaaattgaacacaacaaa

SEQ ID NO: 11 - DNA, 741 bp, synthetic construct
  source 1..741: mol_type = other DNA
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 12 - DNA, 710 bp, synthetic construct
  source 1..710: mol_type = other DNA
ttaaaacagcctgtgggttgatcccacccacagggcccactgggcgtcagcactctggtatcacggtacctttgtgcgcctgttttatactccctccccccaaccgtaacttagaagcagcactaactgatcaatagccagcgcggcatgccagccacgcttcgatcaagcacttctgttaccccggactgagtatcaatagactgctcacgcggttgaaggagaaaacgttcgtcatccggccaactacttcgagaaacctagtaacaccatggaagttgcggagtgtttcgctcagcactaccccagtgtagatcaggtcgatgagtcaccgcattccccacaggcgactgtggcggtggctgcgttggcggcctgcccatggggaaacccatgggacgctcttatatagacatggtgcgaagagtctattgagctagttggtagtcctccggcccctgaatgcggctaatcctaactgcggagcatacaccctcaaaccagagggcagtgtgtcgtaatgggcaactctgcagcggaaccgactactttgggtgtccgtgtttcactttatttctaaattggctgcttatggtgacaattgagagattgttaccatatagctattggattggccatccggtgaccaatagagctattatatatctctttgttgggttcataccacttagcttgaaagaggttaaaac

SEQ ID NO: 13 - DNA, 679 bp, synthetic construct
  source 1..679: mol_type = other DNA
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttgg

SEQ ID NO: 14 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 15 - DNA, 417 bp, synthetic construct
  source 1..417: mol_type = other DNA
agaaaattatttggtgtgagcgattatgatgtaggctcttggattatataaaataattgacccctttgttgaaaggataaatcaacctctttcgttttgaagcgtggaatttcaattcgattgtagtagtgtcattcctatttaggatacggttggacacataaataactaatcttccgcccagctccttgaactataccatctaccactaggagttgtgtaaaattatggtggtcaaaacaacaacaacaattcttcacaatctggcggttcccagtctacctcccccgctcagattcacaatcccgataaaatggtagacaatttgtcaacagacgttcaacaggaaactaatactacgtatttcgaacaacgagcagttttcgatgagacaaccttccaggctcctctctctca

SEQ ID NO: 16 - DNA, 864 bp, synthetic construct
  source 1..864: mol_type = other DNA
aaaatgcgaggcatgggttaccttcccatgccccctccattttaaacaacattatttcaacattttgtgttttatcagactcagtttattgcgttactaattagttttgcgtttatctgcccccgtggtttagcactttatgtgtaaagactcttgattctaaccttgccgtatggtgccctaacgggttcatttaatcatgcgatgagggttgctataccgcatccattctaaggcgattcaatgcttcatttaggaattttgttgacgattaaaaggtacccccacaaaaacaaaaccaatcttacttgattttcgttttaactgaccactgcgatcccaaattttcgccttcttatcaaagtatgttgtgttctttgggtgtacaacctgagaacttgtctacaactacatattactcgaggaagaaattcggtttaagccgtgccttctcacgtttagtatatctatctagacacaccttcttcatcttctaatccccatctagtctcctgatcagagacgtcgttattaacaaataaccccccttgttaataagagacaaagtacaatcaagctaagttctcttggagttcctgtaggaacttagccattgtgatagagtcataagtctatgtgcatagacagctctagctcaccatttccttcccaacccatcttttcatcagcttaactctatgaatccgatgcaaaaaccattctaacatcttatggtgctttccaagccaaatgagagctcactcttttgagccgctatttaatggacaataaacgttttatagtgtacatcatattgtaaaaacaaaatgacaaatacaactaccgaaaacacttacaggaaaa

SEQ ID NO: 17 - DNA, 724 bp, synthetic construct
  source 1..724: mol_type = other DNA
gtaaagactcttgattctaaccttgccgtatggtgccctaacgggttcatttaatcatgcgatgagggttgctataccgcatccattctaaggcgattcaatgcttcatttaggaattttgttgacgattaaaaggtacccccacaaaaacaaaaccaatcttacttgattttcgttttaactgaccactgcgatcccaaattttcgccttcttatcaaagtatgttgtgttctttgggtgtacaacctgagaacttgtctacaactacatattactcgaggaagaaattcggtttaagccgtgccttctcacgtttagtatatctatctagacacaccttcttcatcttctaatccccatctagtctcctgatcagagacgtcgttattaacaaataaccccccttgttaataagagacaaagtacaatcaagctaagttctcttggagttcctgtaggaacttagccattgtgatagagtcataagtctatgtgcatagacagctctagctcaccaaaaaacaaatgttggcctgtatgagagacttcccagactaccggaggtcctcatcaattccaacaaaaacttttaaaacatgtgatcttatcacttttatagtttatgaaatctttattattgttattgctatgttttaaatttctttttcaaaattcatcctgttccagcaatggttttaaaggaacttgtataacaaaaccgcc

SEQ ID NO: 18 - DNA, 259 bp, synthetic construct
  source 1..259: mol_type = other DNA
ccccgtcacctttggcttatcagtggcgcgcctgttggcctgtatgagagacttcccagactaccggaggtcctcatcaattccaacaaaaacttttaaaacatgtgatcttatcacttttatagtttatgaaatctttattattgttattgctatgttttaaatttctttttcaaaattcatcctgttccagcaatggttttaaaggaacttgtataacaaaaccgccccatggagtgacatctggacgctaagaccg

SEQ ID NO: 19 - DNA, 349 bp, synthetic construct
  source 1..349: mol_type = other DNA
ggacttcggtccccctgttactctgcgagccaccgcagagccagggttggtacgcccgaggtgttagaccccggccgaaagctcctaaccattgggttagtaggacgtggtaaatgccactgaggggttggagagctggtagagcgagtaagtcggcgtaaggcccgagtacgggcctcccagcccgggtcagcctaaacctggctgtgatacccggtgcatggagggcgtgtcccaacgctcgatcgctgtagggtgggtccctgcagttgggtgtggctaccctgctcgtactgcttgatagagtcccggcggacggaccagctctcgtcagtccgtggagttgcac

SEQ ID NO: 20 - DNA, 608 bp, synthetic construct
  source 1..608: mol_type = other DNA
ccccgtcacctttggcttatcagtggcgcgccggacttcggtccccctgttactctgcgagccaccgcagagccagggttggtacgcccgaggtgttagaccccggccgaaagctcctaaccattgggttagtaggacgtggtaaatgccactgaggggttggagagctggtagagcgagtaagtcggcgtaaggcccgagtacgggcctcccagcccgggtcagcctaaacctggctgtgatacccggtgcatggagggcgtgtcccaacgctcgatcgctgtagggtgggtccctgcagttgggtgtggctaccctgctcgtactgcttgatagagtcccggcggacggaccagctctcgtcagtccgtggagttgcactgttggcctgtatgagagacttcccagactaccggaggtcctcatcaattccaacaaaaacttttaaaacatgtgatcttatcacttttatagtttatgaaatctttattattgttattgctatgttttaaatttctttttcaaaattcatcctgttccagcaatggttttaaaggaacttgtataacaaaaccgccccatggagtgacatctggacgctaagaccg

SEQ ID NO: 21 - DNA, 741 bp, synthetic construct
  source 1..741: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgagaaacctagtaacaccgtgaaagttgcagagtgtttcgctcagcactaccccagtgtagatcaggtcgatgagtcaccgcattccccacgggcgaccgtggcggtggctgcgctggcggcctgcctacggggaaacccgtaggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 22 - DNA, 742 bp, synthetic construct
  source 1..742: mol_type = other DNA
ttaaaacagcctgtgggttgttcccacccacagggcccattgggcgctagcactctggtattacggtacctttgtgcgcctgttttatgtcccctcccccaatcgcaacttagaagcaacacacactgatcaacagtaagcgtggcataccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacacccccaagccagggggcagtgtgtcgtaacgggtaactctgcagcggaaccgactactttgggtgtccgtgtttcattttattcctatgctggctgcttatggtgacaattgacagattgttaccatatagctattggattggccatccggtgactaatagagccattatataccactttgttgggtttataccactcaacttgaaagaggtcaaaacactacagctcatcattaaattgaacacaacaaa

SEQ ID NO: 23 - DNA, 739 bp, synthetic construct
  source 1..739: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaacgttcgtcatccggccaactacttcgagaaacctagtaacaccatggaagttgcggagtgtttcgctcagcactaccccagtgtagatcaggtcgatgagtcaccgcattccccacaggcgactgtggcggtggctgcgttggcggcctgcccatggggaaacccatgggacgctcttatatagacatggtgcgaagagtctattgagctagttggtagtcctccggcccctgaatgcggtaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 24 - DNA, 712 bp, synthetic construct
  source 1..712: mol_type = other DNA
ttaaaacagcctgtgggttgatcccacccacagggcccactgggcgtcagcactctggtatcacggtacctttgtgcgcctgttttatactccctccccccaaccgtaacttagaagcagcactaactgatcaatagccagcgcggcatgccagccacgcttcgatcaagcacttctgttaccccggactgagtatcaatagactgctcacgcggttgaaggagaaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggcctaatcctaactgcggagcatacaccctcaaaccagagggcagtgtgtcgtaatgggcaactctgcagcggaaccgactactttgggtgtccgtgtttcactttatttctaaattggctgcttatggtgacaattgagagattgttaccatatagctattggattggccatccggtgaccaatagagctattatatatctctttgttgggttcataccacttagcttgaaagaggttaaaac

SEQ ID NO: 25 - DNA, 718 bp, synthetic construct
  source 1..718: mol_type = other DNA
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaattcgttatccggccaactacttcgagaaacctagtaacaccgtgaaagttgcagagtgtttcgctcagcactaccccagtgtagatcaggtcgatgagtcaccgcattccccacgggcgaccgtggcggtggctgcgctggcggcctgcctacggggaaacccgtaggacgctctaatacagacatggtgcgaagagtctattgagctagttggtagtcctccggcccctgaattagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttgg

SEQ ID NO: 26 - DNA, 775 bp, synthetic construct
  source 1..775: mol_type = other DNA
ttaaaacagcctgtgggttgttcccacccacagggcccattgggcgctagcactctggtattacggtacctttgtgcgcctgttttatgtcccctcccccaatcgcaacttagaagcaacacacactgatcaacagtaagcgtggcataccagccacgttttgatcaagcacttctgttaccccggactgagtatcaatagactgctcacgcggttgaaggagcccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggccccttaatcctaactgcggagcacacacccccaagccagggggcagtgtgtcgtaacgggtaactctgcagcggaaccgactactttgggtgtccgtgtttcattttattcctatgctggctgcttatggtgacaattgacagattgttaccatatagctattggattggccatccggtgactaatagagccattatataccactttgttgggtttataccactcaacttgaaagaggtcaaaacactacagctcatcattaaattgaacacaacaaa

SEQ ID NO: 27 - DNA, 3822 bp, synthetic construct
  source 1..3822: mol_type = other DNA
atgttcgttttcctggtgctcctgcctctggtcagcagccagtgcgtgaacctgactactcggacccagctgcctccagcttatacaaattccttcaccagaggcgtttactaccctgacaaggtgttccggagcagcgtgctgcacagcacccaggatctgttcctgcctttcttcagcaacgtgacatggttccacgccatccacgtgtccggcaccaacggcaccaagaggttcgacaaccccgtgctgcctttcaacgacggcgtgtacttcgcctccaccgagaagagcaatatcatcagaggctggatcttcggcaccaccctggactccaagacacagtccctgctgatcgtgaacaacgccaccaacgtggtcatcaaggtgtgtgagttccagttctgtaacgaccccttcctgggcgtgtattaccataagaacaacaagagctggatggaatctgaatttagagtgtacagctctgccaacaactgcacatttgagtacgtgagccagcccttcctgatggacctggagggcaaacagggcaactttaagaacctcagagagttcgtgtttaagaacattgatggatacttcaagatctacagcaagcacacacctatcaacctggtcagggatctgccccagggcttcagcgccctggaacctctggtggacctgcctatcggcatcaacatcaccagattccagaccctgctggccctgcacaggagctacctgacacctggcgacagcagcagcggatggaccgccggcgccgctgcctactacgtgggatacctgcaacctagaacctttctgctgaagtacaatgagaatggcaccatcacagacgccgtggattgcgccctggaccccctgagcgagacaaagtgtacacttaagtctttcacagtggagaagggaatctaccagaccagcaacttccgggtacagcccaccgaaagtatcgtgcggtttccaaacatcaccaacctgtgtcctttcggcgaggtgttcaacgccacccggtttgccagcgtgtacgcctggaacagaaagcggatcagcaactgcgtggccgattactccgtgctgtataacagcgccagcttttctacattcaaatgctacggcgtgtctccaaccaagctcaacgacctgtgcttcaccaacgtgtacgccgattccttcgtcatccggggcgacgaggtgcgccagatcgctcctggccagacaggcaatatcgccgactataactacaaactgcctgatgattttaccggctgtgtgatagcctggaactctaacaacctggattccaaggtgggcggcaattacaactacagatacagactgtttcggaagagcaacctgaagccttttgagagagatatcagcacagaaatctaccaggcgggctccaagccgtgcaacggcgtgaagggctttaattgctatttccctctgcaaagctacggtttccagcctacctacggagttggatatcagccttacagggtggtggtgctgtcattcgagctgttgcatgcccctgccacagtgtgcggacctaagaaatccaccaacctggtgaagaacaagtgcgtgaactttaattttaacggcctgaccggcaccggcgtgctgaccgagagcaacaagaagtttctgcccttccaacaatttggcagagacatcgctgacaccacagacgccgtcagagatccccaaaccctggaaatcctggacatcacaccttgttctttcggcggagtcagcgtgatcacacccggcaccaacacctctaatcaagtggccgtgctgtaccagggcgtgaactgcaccgaggtgcccgtcgccatccacgccgaccagctgacacccacatggcgggtgtacagcacaggaagcaatgtcttccagaccagagccggctgcctgatcggagccgaacacgtgaacaacagctacgaatgtgacattcccatcggcgctggcatctgtgccagctaccagacacaaaccaactctcctggttccgccagcagcgttgctagccagagcatcatcgcttataccatgagcctgggcgcagaaaactccgtggcctacagcaacaacagcatcgccatacccaccaacttcacaatctcagtgacaacggaaatcctgcctgtgtctatgacaaagacctctgtggattgtaccatgtacatctgcggagattctacagaatgctccaaccttctgcttcagtatggcagcttctgcacccagctgaatagagccctgaccggcatcgccgtggagcaggacaagaacacccaggaagtcttcgcccaggtcaaacagatctacaagacccctccaatcaaggacttcggtggctttaacttcagccagatcctgcctgatccttcaaagccgagcaaaagaagccctatcgaggacctgctattcaacaaggtgacccttgccgacgccggcttcatcaagcagtacggtgactgcctgggcgacattgctgctagagacttgatctgcgcccagaaattcaacggcctgacagtgctgcctcctctgctgaccgatgagatgatcgctcagtacacaagtgccctcctggccggcaccatcactagcggatggacattcggcgccggccctgccctgcaaatcccattccccatgcagatggcctacagattcaatggaatcggcgtgacccaaaacgtactgtacgagaaccaaaaactgatcgcaaaccagttcaattctgccatcggcaagatccaggacagcctgagttctacccccagcgccctgggaaagctgcaagacgtggtgaaccagaatgctcaggccctgaataccctggtgaaacagctgagctccaacttcggcgccatcagctctgtgctgaatgacatcctgtctagactggaccctccagaggccgaggtgcagatcgatcggctcatcaccggaagactgcaaagcctgcaaacatacgtgacccagcagctcatcagagccgccgagatccgggccagcgccaatctcgctgctacaaagatgagcgagtgtgtgctgggacagtctaagcgggtcgacttctgcggcaaaggctaccacctgatgagctttccccagagcgccccccacggcgtggtattcctgcacgtgacctacgttcctgcccaggagaaaaattttacaacagctcctgcgatctgccacgacggcaaggcccacttccccagagaaggcgtgttcgtgagcaacggcacacactggttcgtgacccaaagaaacttctacgaacctcaaatcatcaccactgataacacattcgtgtctggcaactgtgacgtggtgatcggcatcgtgaacaataccgtatacgacccactgcaacctgagctcgattccttcaaagaggaactggacaagtacttcaagaaccacacctctcctgacgtggacctgggagatatcagcggcatcaacgccagcgtggtgaacatccaaaaggaaatcgacagactgaacgaggtagccaagaacctgaacgagagcctgattgacctgcaagagctgggcaagtacgagcagtacatcaagtggccttggtacatctggctgggattcatcgccggcctgattgccatcgtgatggtgaccatcatgctgtgctgcatgaccagctgctgtagctgtctgaaaggctgctgtagctgcggcagctgctgtaaattcgacgaggacgacagcgagcccgtgctgaagggcgtgaagctgcactacacctga

SEQ ID NO: 28 - DNA, 3795 bp, synthetic construct
  source 1..3795: mol_type = other DNA
atgttcgtgttcctggtgctcctgcccctggtgtccagccagtgcgtgaacctgacaacacggacccagctgcctcctgcctacacaaattcttttaccagaggtgtttactaccccgacaaggtgttccggagctctgtactgcacagcacgcaggacctctttctgccttttttcagcaacgtgacctggttccacgccatccacgtgagtggcaccaacggcaccaagcggtttgataatcctgtgctgcccttcaacgacggcgtgtacttcgcctccaccgagaagagcaatatcatccggggctggatcttcggcacaaccctggacagcaaaacgcagtctctgctgatcgtgaacaacgccaccaatgtggtgatcaaggtgtgcgagttccaattttgtaatgaccccttcctgggcgtgtactaccacaagaacaacaaaagctggatggaaagcgagttccgggtctacagcagcgccaacaactgcacattcgagtacgtgagccagccctttctgatggatctggaaggaaaacaggggaacttcaaaaacctgagagagttcgtgttcaagaacattgacggctactttaagatctatagcaagcacacaccaatcaatctcgtgcgggatctgcctcagggcttcagcgccctggaacccctggtggaccttcctatcggaatcaatatcactagattccaaacattgctggccctgcacagatcttatctgacacctggagattcttcaagcggctggaccgccggcgccgctgcatactatgtgggctacctgcaacctcggaccttcctgttaaagtacaacgagaacggcaccattacagatgccgtggattgcgccctggaccctctgtctgaaaccaagtgtacactgaaaagcttcaccgtggaaaagggaatctaccagaccagcaacttcagagtgcagcctaccgagagcatcgtcagatttcctaacatcacaaacctgtgccccttcggcgaggtgtttaatgccacccggttcgcctccgtgtatgcctggaaccggaagcggatctctaactgcgtcgccgactactctgtgctgtacaactccgccagcttctccacattcaagtgctacggcgtgtcccctaccaagctgaacgacctgtgcttcaccaacgtgtacgccgatagtttcgtcatccggggcgacgaggtgcggcaaatcgctcctggccagaccggcaacatcgctgactacaactacaagttgcccgatgatttcaccggctgcgtgatcgcctggaactccaacaacctggactctaaggttggcggaaactacaactacagatacagactgttcaggaagagcaatctgaaacctttcgaaagagacatctctaccgagatctaccaggctggctccaaaccctgcaacggcgtcaagggcttcaactgctacttccccctgcaaagctacggattccagcccacctatggcgtgggctaccagccttacagagtggttgtcctgagcttcgagctgcttcacgctccagccacagtgtgcggccccaagaagtctacaaacctggtgaagaacaagtgcgtcaacttcaacttcaacggcctgacaggcacaggtgtgctgaccgaaagcaacaaaaagtttctgccttttcagcagttcggcagggatatcgccgacaccactgacgccgtgagggaccctcagacactggaaattctggacatcaccccttgcagctttggtggggtgagcgtgatcactcccggcaccaatacctcgaaccaggtggctgttctgtaccaaggcgtgaactgtaccgaggtgcccgtggccatccacgccgaccagctgactccgacctggagagtgtacagcacaggctctaatgtgtttcaaacgagagccggctgtctgatcggtgcggagcacgtgaacaactcctacgagtgcgacatcccaatcggcgctggaatctgcgcctcttaccagggcggaagcggcggaagcagcatcatcgcctacaccatgagcctgggcgctgagaacagtgtggcttgtagtaacaattctatcgccattcccacaaacttcaccatcagcgtgaccaccgaaatcctgcctgtcagcatgaccaagacaagcgtggactgcaccatgtacatctgcggcgactctaccgagtgcagcaacctgctgctgcaatacggctccttctgcacccagctgaatagagctctgaccggaatcgccgtcgagcaggataagaacacacaggaggtgttcgcccaggttaagcagatctacaagacccctccaattaaggacttcggcggctttaacttcagccagattctgcccgacccctctaaacctagcaagagaagccctatcgaggacctgctgttcaacaaggtgaccctggctgacgccggcttcatcaagcagtacggcgattgtctgggcgatatcgcagccagagacctgatctgtgcccagaagttcaacggcttaaccgtgctgcctcctctcctcaccgatgagatgatcgcccagtataccagcgccctgctggccggaaccatctgtagcggctggacgttcggcgccggccctgccctgcaaatcccttttccaatgcagatggcctatagatttaacggcatcggagtgacccaaaacgtgctgtacgagaatcagaaactgatcgccaaccagttcaacagcgctatcggcaagatccaagactccctgtcctctacacccagcgcccttggcaagctgcaagacgtggtgaaccagaacgctcaggctctgaacacactggtgaagcagctgagctccaatttcggcgccatcagctcggtgctcaacgacatcctgagtagactggacaagccagaggccgaggtgcagatcgatagactgatcacaggcagactgcaaagcctgcaaacctacgtgacacaacagctcatcagagccgccgaaattagagccagcgccaatctggccgctacaaaaatgagcgaatgcgtgctgggacagagcaagcgcgttgatttctgtggcaagggctatcacctgatgagctttcctcagagcgcccctcatggcgtggtgttcctgcacgtgacttacgtacctgcacaggagaaaaactttaccaccgcccctgccatctgtcacgacggcaaagcccacttccccagggagggcgtgttcgtgtccaacggcacccactggttcgtgacacagcgaaacttctacgagcctcagatcatcaccacagacaacaccttcgtgagtggcaattgcgacgtggtgatcggcattgtgaacaacaccgtgtacgaccccctgcaaccagaactggatagcttcaaagaagagctggataagtacttcaagaaccacaccagccctgacgtggatctgggcgacatcagcggcattaatgccagcgtggtaaacatccagaaggaaatcgacagactgaacgaggtggccaagaacctgaatgagagcctgatagacttgcaggagctgggaaagtatgaacaatacatcaagtggccttggtacatctggctgggcttcatcgccggcctgatcgctatcgtgatggtgacaatcatgctgtgctgcatgacatcctgttgcagctgcctgaagggctgctgttcttgtggcagctgctgcaagttcgacgaagatgactctgaacccgtgcttaaaggcgtgaagctgcattacacctga

SEQ ID NO: 29 - DNA, 522 bp, synthetic construct
  source 1..522: mol_type = other DNA
atgagagtgacagcccctagaacactgattctgctgctgagcggagccctggccctgacagaaacatgggccggaagcggcggatctggcggaggcggatctggcggaaaagcacccaggaagcaactggctacaaaagccgctcgcatgagtgcgccctctactggaggggtgaagaaacctcatcgtggaggatctggaggaggaggatctggaggacagtacatcaaggccaacagcaagttcatcggcatcaccgagctgatgaccaacagcgtggacgacgccctgatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagcctaa

SEQ ID NO: 30 - DNA, 666 bp, synthetic construct
  source 1..666: mol_type = other DNA
atgagagtgacagcccctagaacactgattctgctgctgagcggagccctggccctgacagaaacatgggccggaagcggcggatctggcggaggcggatctggcggactgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagctccggaggatctggaggaggaggatctggaggacagtacatcaaggccaacagcaagttcatcggcatcaccgagctgatgaccaacagcgtggacgacgccctcatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagcc

SEQ ID NO: 31 - DNA, 1173 bp, synthetic construct
  source 1..1173: mol_type = other DNA
atgagagtgacagcccctagaacactgattctgctgctgagcggagccctggccctgacagaaacatgggccggaagcggcggatctggcggaggcggatctggcggacaggacaactgcacctgcccaaccaacaagatgaccgtgtgttccccagacggaccaggaggacgctgccagtgccgcgccctcggaagtggaatggctgtggactgctccacccttaccagcaagtgccttcttcttaaggcccggatgtccgccccaaagaacgccaggaccctcgtgcggccaagcgagcacgcccttgtggacaacgacggcctttacgacccagactgcgacccagagggccggtttaaggccaggcagtgcaaccagaccagcgtgtgctggtgcgtgaacagcgtgggcgtgcggcggaccgacaagggcgaccttagccttcggtgcgacgagcttgtgcggacccaccacatccttatcgaccttcggcaccggccaaccgccggcgcctttaaccactccgaccttgacgccgagcttaggaggctctttagggaaaggtacagacttcacccaaagtttgtggccgccgtgcactacgagcagccaaccatccagatcgagcttcggcagaacaccagccagaaggccgccggcgacgtggacatcggcgacgccgcctactactttgagagagacatcaagggcgagagcctttttcagggcagaggcggccttgaccttagagtgcggggcgagccacttcaggtggagagaacccttatctactaccttgacgagatcccaccaaagttttccatgaagagacttaccggaggatctggaggaggaggatctggaggacagtacatcaaggccaacagcaagttcatcggcatcaccgagctgatgaccaacagcgtggacgacgccctgatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagcctaa

SEQ ID NO: 32 - DNA, 4959 bp, synthetic construct
  source 1..4959: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacgatatccagtgtactaattacgctttgttgaaactcgctggcgatgttgaaagtaaccccggtcctatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggatccgagggcagaggaagtctgctaacatgcggtgacgtcgaggagaatcctggcccaatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgagatccgtgaaacagactttgaattttgaccttctcaagttggcgggagacgtggagtccaacccagggcccatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaa

SEQ ID NO: 33 - DNA, 1425 bp, synthetic construct
  source 1..1425: mol_type = other DNA
atggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggctccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatccggagcgcaaatgttgtgtcgagtgcccaccgtgcccaaaaaagcttaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 34 - DNA, 1656 bp, synthetic construct
  source 1..1656: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaactcgagtaa

SEQ ID NO: 35 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtga

SEQ ID NO: 36 - DNA, 18 bp, synthetic construct
  source 1..18: mol_type = other DNA
aaaaaacaaaaaacaaaa

SEQ ID NO: 37 - DNA, 22 bp, synthetic construct
  source 1..22: mol_type = other DNA
cggctattatgcgttaccggcg

SEQ ID NO: 38 - DNA, 123 bp, synthetic construct
  source 1..123: mol_type = other DNA
agacgctacggacttaaataattgagccttaaagaagaaattctttaagtggatgctctcaaactcagggaaacctaaatctagttatagacaaggcaatcctgagccaagccgaagtagtaa

SEQ ID NO: 39 - skipped

SEQ ID NO: 40 - DNA, 21 bp, synthetic construct
  source 1..21: mol_type = other DNA
gagccgtgtttgtatccaatc

SEQ ID NO: 41 - DNA, 27 bp, synthetic construct
  source 1..27: mol_type = other DNA
taggattctcattgctgggatccctgg

SEQ ID NO: 42 - DNA, 4008 bp, synthetic construct
  source 1..4008: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtgcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 43 - DNA, 7830 bp, synthetic construct
  source 1..7830: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatgttcgttttcctggtgctcctgcctctggtcagcagccagtgcgtgaacctgactactcggacccagctgcctccagcttatacaaattccttcaccagaggcgtttactaccctgacaaggtgttccggagcagcgtgctgcacagcacccaggatctgttcctgcctttcttcagcaacgtgacatggttccacgccatccacgtgtccggcaccaacggcaccaagaggttcgacaaccccgtgctgcctttcaacgacggcgtgtacttcgcctccaccgagaagagcaatatcatcagaggctggatcttcggcaccaccctggactccaagacacagtccctgctgatcgtgaacaacgccaccaacgtggtcatcaaggtgtgtgagttccagttctgtaacgaccccttcctgggcgtgtattaccataagaacaacaagagctggatggaatctgaatttagagtgtacagctctgccaacaactgcacatttgagtacgtgagccagcccttcctgatggacctggagggcaaacagggcaactttaagaacctcagagagttcgtgtttaagaacattgatggatacttcaagatctacagcaagcacacacctatcaacctggtcagggatctgccccagggcttcagcgccctggaacctctggtggacctgcctatcggcatcaacatcaccagattccagaccctgctggccctgcacaggagctacctgacacctggcgacagcagcagcggatggaccgccggcgccgctgcctactacgtgggatacctgcaacctagaacctttctgctgaagtacaatgagaatggcaccatcacagacgccgtggattgcgccctggaccccctgagcgagacaaagtgtacacttaagtctttcacagtggagaagggaatctaccagaccagcaacttccgggtacagcccaccgaaagtatcgtgcggtttccaaacatcaccaacctgtgtcctttcggcgaggtgttcaacgccacccggtttgccagcgtgtacgcctggaacagaaagcggatcagcaactgcgtggccgattactccgtgctgtataacagcgccagcttttctacattcaaatgctacggcgtgtctccaaccaagctcaacgacctgtgcttcaccaacgtgtacgccgattccttcgtcatccggggcgacgaggtgcgccagatcgctcctggccagacaggcaatatcgccgactataactacaaactgcctgatgattttaccggctgtgtgatagcctggaactctaacaacctggattccaaggtgggcggcaattacaactacagatacagactgtttcggaagagcaacctgaagccttttgagagagatatcagcacagaaatctaccaggcgggctccaagccgtgcaacggcgtgaagggctttaattgctatttccctctgcaaagctacggtttccagcctacctacggagttggatatcagccttacagggtggtggtgctgtcattcgagctgttgcatgcccctgccacagtgtgcggacctaagaaatccaccaacctggtgaagaacaagtgcgtgaactttaattttaacggcctgaccggcaccggcgtgctgaccgagagcaacaagaagtttctgcccttccaacaatttggcagagacatcgctgacaccacagacgccgtcagagatccccaaaccctggaaatcctggacatcacaccttgttctttcggcggagtcagcgtgatcacacccggcaccaacacctctaatcaagtggccgtgctgtaccagggcgtgaactgcaccgaggtgcccgtcgccatccacgccgaccagctgacacccacatggcgggtgtacagcacaggaagcaatgtcttccagaccagagccggctgcctgatcggagccgaacacgtgaacaacagctacgaatgtgacattcccatcggcgctggcatctgtgccagctaccagacacaaaccaactctcctggttccgccagcagcgttgctagccagagcatcatcgcttataccatgagcctgggcgcagaaaactccgtggcctacagcaacaacagcatcgccatacccaccaacttcacaatctcagtgacaacggaaatcctgcctgtgtctatgacaaagacctctgtggattgtaccatgtacatctgcggagattctacagaatgctccaaccttctgcttcagtatggcagcttctgcacccagctgaatagagccctgaccggcatcgccgtggagcaggacaagaacacccaggaagtcttcgcccaggtcaaacagatctacaagacccctccaatcaaggacttcggtggctttaacttcagccagatcctgcctgatccttcaaagccgagcaaaagaagccctatcgaggacctgctattcaacaaggtgacccttgccgacgccggcttcatcaagcagtacggtgactgcctgggcgacattgctgctagagacttgatctgcgcccagaaattcaacggcctgacagtgctgcctcctctgctgaccgatgagatgatcgctcagtacacaagtgccctcctggccggcaccatcactagcggatggacattcggcgccggccctgccctgcaaatcccattccccatgcagatggcctacagattcaatggaatcggcgtgacccaaaacgtactgtacgagaaccaaaaactgatcgcaaaccagttcaattctgccatcggcaagatccaggacagcctgagttctacccccagcgccctgggaaagctgcaagacgtggtgaaccagaatgctcaggccctgaataccctggtgaaacagctgagctccaacttcggcgccatcagctctgtgctgaatgacatcctgtctagactggaccctccagaggccgaggtgcagatcgatcggctcatcaccggaagactgcaaagcctgcaaacatacgtgacccagcagctcatcagagccgccgagatccgggccagcgccaatctcgctgctacaaagatgagcgagtgtgtgctgggacagtctaagcgggtcgacttctgcggcaaaggctaccacctgatgagctttccccagagcgccccccacggcgtggtattcctgcacgtgacctacgttcctgcccaggagaaaaattttacaacagctcctgcgatctgccacgacggcaaggcccacttccccagagaaggcgtgttcgtgagcaacggcacacactggttcgtgacccaaagaaacttctacgaacctcaaatcatcaccactgataacacattcgtgtctggcaactgtgacgtggtgatcggcatcgtgaacaataccgtatacgacccactgcaacctgagctcgattccttcaaagaggaactggacaagtacttcaagaaccacacctctcctgacgtggacctgggagatatcagcggcatcaacgccagcgtggtgaacatccaaaaggaaatcgacagactgaacgaggtagccaagaacctgaacgagagcctgattgacctgcaagagctgggcaagtacgagcagtacatcaagtggccttggtacatctggctgggattcatcgccggcctgattgccatcgtgatggtgaccatcatgctgtgctgcatgaccagctgctgtagctgtctgaaaggctgctgtagctgcggcagctgctgtaaattcgacgaggacgacagcgagcccgtgctgaagggcgtgaagctgcactacacctgagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - DNA, 7803 bp, synthetic construct
  source 1..7803: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatgttcgtgttcctggtgctcctgcccctggtgtccagccagtgcgtgaacctgacaacacggacccagctgcctcctgcctacacaaattcttttaccagaggtgtttactaccccgacaaggtgttccggagctctgtactgcacagcacgcaggacctctttctgccttttttcagcaacgtgacctggttccacgccatccacgtgagtggcaccaacggcaccaagcggtttgataatcctgtgctgcccttcaacgacggcgtgtacttcgcctccaccgagaagagcaatatcatccggggctggatcttcggcacaaccctggacagcaaaacgcagtctctgctgatcgtgaacaacgccaccaatgtggtgatcaaggtgtgcgagttccaattttgtaatgaccccttcctgggcgtgtactaccacaagaacaacaaaagctggatggaaagcgagttccgggtctacagcagcgccaacaactgcacattcgagtacgtgagccagccctttctgatggatctggaaggaaaacaggggaacttcaaaaacctgagagagttcgtgttcaagaacattgacggctactttaagatctatagcaagcacacaccaatcaatctcgtgcgggatctgcctcagggcttcagcgccctggaacccctggtggaccttcctatcggaatcaatatcactagattccaaacattgctggccctgcacagatcttatctgacacctggagattcttcaagcggctggaccgccggcgccgctgcatactatgtgggctacctgcaacctcggaccttcctgttaaagtacaacgagaacggcaccattacagatgccgtggattgcgccctggaccctctgtctgaaaccaagtgtacactgaaaagcttcaccgtggaaaagggaatctaccagaccagcaacttcagagtgcagcctaccgagagcatcgtcagatttcctaacatcacaaacctgtgccccttcggcgaggtgtttaatgccacccggttcgcctccgtgtatgcctggaaccggaagcggatctctaactgcgtcgccgactactctgtgctgtacaactccgccagcttctccacattcaagtgctacggcgtgtcccctaccaagctgaacgacctgtgcttcaccaacgtgtacgccgatagtttcgtcatccggggcgacgaggtgcggcaaatcgctcctggccagaccggcaacatcgctgactacaactacaagttgcccgatgatttcaccggctgcgtgatcgcctggaactccaacaacctggactctaaggttggcggaaactacaactacagatacagactgttcaggaagagcaatctgaaacctttcgaaagagacatctctaccgagatctaccaggctggctccaaaccctgcaacggcgtcaagggcttcaactgctacttccccctgcaaagctacggattccagcccacctatggcgtgggctaccagccttacagagtggttgtcctgagcttcgagctgcttcacgctccagccacagtgtgcggccccaagaagtctacaaacctggtgaagaacaagtgcgtcaacttcaacttcaacggcctgacaggcacaggtgtgctgaccgaaagcaacaaaaagtttctgccttttcagcagttcggcagggatatcgccgacaccactgacgccgtgagggaccctcagacactggaaattctggacatcaccccttgcagctttggtggggtgagcgtgatcactcccggcaccaatacctcgaaccaggtggctgttctgtaccaaggcgtgaactgtaccgaggtgcccgtggccatccacgccgaccagctgactccgacctggagagtgtacagcacaggctctaatgtgtttcaaacgagagccggctgtctgatcggtgcggagcacgtgaacaactcctacgagtgcgacatcccaatcggcgctggaatctgcgcctcttaccagggcggaagcggcggaagcagcatcatcgcctacaccatgagcctgggcgctgagaacagtgtggcttgtagtaacaattctatcgccattcccacaaacttcaccatcagcgtgaccaccgaaatcctgcctgtcagcatgaccaagacaagcgtggactgcaccatgtacatctgcggcgactctaccgagtgcagcaacctgctgctgcaatacggctccttctgcacccagctgaatagagctctgaccggaatcgccgtcgagcaggataagaacacacaggaggtgttcgcccaggttaagcagatctacaagacccctccaattaaggacttcggcggctttaacttcagccagattctgcccgacccctctaaacctagcaagagaagccctatcgaggacctgctgttcaacaaggtgaccctggctgacgccggcttcatcaagcagtacggcgattgtctgggcgatatcgcagccagagacctgatctgtgcccagaagttcaacggcttaaccgtgctgcctcctctcctcaccgatgagatgatcgcccagtataccagcgccctgctggccggaaccatctgtagcggctggacgttcggcgccggccctgccctgcaaatcccttttccaatgcagatggcctatagatttaacggcatcggagtgacccaaaacgtgctgtacgagaatcagaaactgatcgccaaccagttcaacagcgctatcggcaagatccaagactccctgtcctctacacccagcgcccttggcaagctgcaagacgtggtgaaccagaacgctcaggctctgaacacactggtgaagcagctgagctccaatttcggcgccatcagctcggtgctcaacgacatcctgagtagactggacaagccagaggccgaggtgcagatcgatagactgatcacaggcagactgcaaagcctgcaaacctacgtgacacaacagctcatcagagccgccgaaattagagccagcgccaatctggccgctacaaaaatgagcgaatgcgtgctgggacagagcaagcgcgttgatttctgtggcaagggctatcacctgatgagctttcctcagagcgcccctcatggcgtggtgttcctgcacgtgacttacgtacctgcacaggagaaaaactttaccaccgcccctgccatctgtcacgacggcaaagcccacttccccagggagggcgtgttcgtgtccaacggcacccactggttcgtgacacagcgaaacttctacgagcctcagatcatcaccacagacaacaccttcgtgagtggcaattgcgacgtggtgatcggcattgtgaacaacaccgtgtacgaccccctgcaaccagaactggatagcttcaaagaagagctggataagtacttcaagaaccacaccagccctgacgtggatctgggcgacatcagcggcattaatgccagcgtggtaaacatccagaaggaaatcgacagactgaacgaggtggccaagaacctgaatgagagcctgatagacttgcaggagctgggaaagtatgaacaatacatcaagtggccttggtacatctggctgggcttcatcgccggcctgatcgctatcgtgatggtgacaatcatgctgtgctgcatgacatcctgttgcagctgcctgaagggctgctgttcttgtggcagctgctgcaagttcgacgaagatgactctgaacccgtgcttaaaggcgtgaagctgcattacacctgagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 45 - DNA, 4530 bp, synthetic construct
  source 1..4530: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatgagagtgacagcccctagaacactgattctgctgctgagcggagccctggccctgacagaaacatgggccggaagcggcggatctggcggaggcggatctggcggaaaagcacccaggaagcaactggctacaaaagccgctcgcatgagtgcgccctctactggaggggtgaagaaacctcatcgtggaggatctggaggaggaggatctggaggacagtacatcaaggccaacagcaagttcatcggcatcaccgagctgatgaccaacagcgtggacgacgccctgatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagcctaagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 4674 bp, synthetic construct
  source 1..4674: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatgagagtgacagcccctagaacactgattctgctgctgagcggagccctggccctgacagaaacatgggccggaagcggcggatctggcggaggcggatctggcggactgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagctccggaggatctggaggaggaggatctggaggacagtacatcaaggccaacagcaagttcatcggcatcaccgagctgatgaccaacagcgtggacgacgccctcatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagccgcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 47 - DNA, 5181 bp, synthetic construct
  source 1..5181: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatgagagtgacagcccctagaacactgattctgctgctgagcggagccctggccctgacagaaacatgggccggaagcggcggatctggcggaggcggatctggcggacaggacaactgcacctgcccaaccaacaagatgaccgtgtgttccccagacggaccaggaggacgctgccagtgccgcgccctcggaagtggaatggctgtggactgctccacccttaccagcaagtgccttcttcttaaggcccggatgtccgccccaaagaacgccaggaccctcgtgcggccaagcgagcacgcccttgtggacaacgacggcctttacgacccagactgcgacccagagggccggtttaaggccaggcagtgcaaccagaccagcgtgtgctggtgcgtgaacagcgtgggcgtgcggcggaccgacaagggcgaccttagccttcggtgcgacgagcttgtgcggacccaccacatccttatcgaccttcggcaccggccaaccgccggcgcctttaaccactccgaccttgacgccgagcttaggaggctctttagggaaaggtacagacttcacccaaagtttgtggccgccgtgcactacgagcagccaaccatccagatcgagcttcggcagaacaccagccagaaggccgccggcgacgtggacatcggcgacgccgcctactactttgagagagacatcaagggcgagagcctttttcagggcagaggcggccttgaccttagagtgcggggcgagccacttcaggtggagagaacccttatctactaccttgacgagatcccaccaaagttttccatgaagagacttaccggaggatctggaggaggaggatctggaggacagtacatcaaggccaacagcaagttcatcggcatcaccgagctgatgaccaacagcgtggacgacgccctgatcaacagcaccaagatctacagctacttccccggaggatctctgggaggaggaggatctggaattgtgggcatcgtggccggcctggccgtgctggccgtggtggtgatcggcgccgtggtggccaccgtgatgtgccggagaaagagcagcggcggcaagggaggaagctacagccaggccgccagcagcgacagcgcccagggatctgatgtgagcctgacagcctaagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 8967 bp, synthetic construct
  source 1..8967: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaacgatatccagtgtactaattacgctttgttgaaactcgctggcgatgttgaaagtaaccccggtcctatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgggatccgagggcagaggaagtctgctaacatgcggtgacgtcgaggagaatcctggcccaatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgagatccgtgaaacagactttgaattttgaccttctcaagttggcgggagacgtggagtccaacccagggcccatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 49 - DNA, 5433 bp, synthetic construct
  source 1..5433: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgctaatacgactcactatagggagaccctcgagattggatacaaacacggctcagatctaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccggggcccaaaaacaaaaaacaaaaaaaacaaaaaaaaaaccaaaaaaacaaaacacagcatgccacaggcgcgccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaccggtatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggctccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcctcgtacgtcaccgtctcttcacaggatccggagcgcaaatgttgtgtcgagtgcccaccgtgcccaaaaaagcttaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaagcggccgcaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagagccgtgtttgtatccaatcataacagcatatctagaatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc